(19) United States
(12) Patent Application Publication
(10) Pub. No.: US 2025/0236863 A1
(43) Pub. Date: Jul. 24, 2025

(54) High Throughput Assays for Identifying the Biological Response of a Cell

(71) Applicant: Zafrens Inc., San Diego, CA (US)

(72) Inventor: Kandaswamy Vijayan, San Diego, CA (US)

(73) Assignee: Zafrens Inc., San Diego, CA (US)

(21) Appl. No.: 19/035,214

(22) Filed: Jan. 23, 2025

Related U.S. Application Data
(60) Provisional application No. 63/624,167, filed on Jan. 23, 2024.

(51) Int. Cl. C12N 15/10

(52) U.S. Cl. CPC C12N 15/1065

(57) ABSTRACT

Methods and compositions allow for high throughput cellular analysis across multiple parameters simultaneously. The methods allow for the mass screening and mass harvesting of data with regard to the response of large numbers of cells to a large number of drug products or other biological stimuli. Microbeads or microparticles carrying multiple biological components may be used to introduce the drug products or biological stimuli. Perturbation beads deliver a perturbation agent and a perturbation barcode, spatial-index beads deliver identifying barcodes, and capture beads capture an analyte produced by a stimulated cell. The methods allow the user to identify a specific biological response of a specific cell in a biochip or other cellular analysis vessel to a specific perturbation agent.

### CROSS-REFERENCE TO RELATED APPLICATION(S)

This application claims priority from U.S. Provisional Patent Application No. 63/624,167, filed Jan. 23, 2024, titled “High Throughput Assays for Identifying the Biological Response of a Cell,” the entire contents of which are hereby fully incorporated herein by reference for all purposes.

### TECHNICAL FIELD

The invention relates to high throughput cellular analysis and the relating of collected cellular genomic information to functional and imaging analysis performed on the same cell, and compositions for conducting same.

### BACKGROUND

Despite advances in research and technology of single-cell analysis, there continues to be a gap between correlating functional cellular data and molecular cellular data for cells at very high throughput. Conventional single cell analysis provides information on genetic profiles of cells and some select proteins, but not their functional state. Relating the functional characteristics of individual cells to their underlying molecular constitution is therefore very valuable for understanding, modeling, and genetically engineering biology. Techniques to render the association between molecular and functional states of cells at scale are difficult because of the wide variety of analytes that need to be profiled, as well as the unavailability to spatially trace defined functional assays to pooled sequencing readouts. In conventional single-cell sequencing approaches, genomic content from individual cells is pooled together before loading onto a next-generation deoxyribonucleic acid (DNA) sequencer. The molecular state of individual cells may be resolved through DNA analysis that tracks nucleic acid content derived from individual cells. But these analyses do not track the positional or temporal information of assays or perturbations these cells may have undergone, rendering it impossible to assign functional characteristics to the molecular profiles compiled by such single-cell sequencing methods.

There thus remains a need for an efficient and effective method for systematically correlating the genomic information of a cell with its functional information obtained from assays at high throughput.

### SUMMARY

The invention provides methods and compositions for high throughput cellular analysis across multiple parameters simultaneously. In one embodiment the methods allow for the mass screening and mass collection of data with regard to the response of cells to drug candidates, small molecules, biomolecules, or other stimuli. In some embodiments the methods involve the use of microbeads carrying multiple chemical or biological components. In one embodiment the methods involve the use of perturbation microbeads to deliver a perturbation agent and perturbation barcodes, spatial-index microbeads to deliver identifying spatial index barcodes, and capture microbeads to capture an analyte produced by a stimulated cell and barcodes released by other microbeads present in the same compartment. The methods allow the user to identify and quantify a specific biological response of a specific cell to a specific perturbation agent.

In a first aspect the invention provides methods for identifying a biological response of a cell to a perturbation agent. The methods can involve one or more of the following steps: a) dispensing cells into a plurality of examination areas present on a biochip, b) dispensing a perturbation agent and a perturbation barcode into the plurality of examination areas, c) dispensing spatial index microbeads into the plurality of examination areas, where the spatial index microbeads have optical labels and spatial-index barcodes that are releasable from the spatial index microbeads, and the spatial index microbeads do not have a bead type identifier sequence; d) dispensing capture microbeads into the plurality of examination areas, the capture microbeads having binding elements for binding the perturbation barcodes, and binding elements for binding the spatial-index barcodes, and further having a capture material that binds to an analyte produced by the cells; e) imaging the biochip to identify the locations of the spatial index microbeads; f) releasing an analyte from the plurality of cells; g) releasing the spatial-index barcodes from the spatial index microbeads, h) binding the released perturbation barcodes and the released spatial-index barcodes to the respective binding elements on the capture microbeads for binding the perturbation barcodes and spatial index barcodes, i) binding the released analyte to the capture material on the capture microbead; j) at least partially sequencing the bound perturbation barcodes and the bound spatial-index barcodes, or their complement sequences; k) identifying the analyte; and l) correlating the analyte, the at least partially sequenced perturbation barcodes and spatial index barcodes, or their complement sequences, with the optical imaging to thereby identify a biological response of a cell to a perturbation agent.

In one embodiment the perturbation agent and perturbation barcodes are present on the capture microbeads or on perturbation microbeads, and the perturbation barcodes have an oligonucleotide sequence, and the perturbation agent and barcodes are dispensed by releasing them from the capture microbeads or perturbation microbeads. The perturbation agent and perturbation barcodes can be present on perturbation microbeads, and the perturbation barcodes can have an oligonucleotide sequence, and the perturbation agent and barcodes can be dispensed by releasing them from the perturbation microbeads. In one embodiment the spatial index barcodes are oligonucleotide sequences. The biochip can have at least 9,000 examination areas having a volume of 1 nanoliter or greater. The cells can be dispensed so that at least 50% of the plurality of examination areas contain a single cell. In any embodiment the capture microbeads do not have a bead type identifier (BID) sequence. The methods can not involve (or can omit) a step of the sequencing of a bead type identifier sequence.

In some embodiments the methods can involve a step of incubating the cells in the examination areas for at least 8 hours. In any embodiment the spatial index microbeads can be selected from a bead library of at least 100 or 250 beads that each have a distinct optical label. The spatial index microbeads can contain fluorescent dyes having an emission peak at a wavelength of 700 um or greater. In any embodiment at least 50% of the examination areas have more than one cell from the plurality of cells. The spatial index microbeads in a well can provide a combination of optical labels that is unique relative to any other examination area in the plurality of examination areas. In any embodiment the spatial index beads can be chosen from a bead library having at least 100 distinct beads. The spatial index barcodes can be an oligonucleotide sequence of no more than 150 nucleotides. The spatial index barcodes can be oligonucleotides, and the binding elements and capture material can be present on on a capture group that contains a unique molecular identifier sequence.

In any embodiment the optical label can be a fluorescent dye that has a color code. The examination areas on the biochip can be identified based on the locations of the color codes from the spatial index microbeads. The color codes can be correlated to a specific examination area on an x-y gradient on the biochip. In any embodiment more than one spatial index microbead can be present in at least 50% of the examination areas. The methods can involve a step of dispensing at least three capture microbeads into a majority of the examination areas. In any embodiment the examination areas can be at least 110 um in diameter. In any embodiment the spatial index beads and perturbation beads can be 20-50 um in diameter. And in any embodiment the capture microbeads can be at least 75 um in diameter. The methods can involve a step of dispensing more than one cell to each examination area. In various embodiments the perturbation agent can be a small molecule drug candidate, or a nucleic acid sequence.

In any embodiment the perturbation agent and the perturbation barcode can be released from the perturbation microbead by photocleavage; and in any embodiment the spatial-index barcodes can be released from the spatial-index microbeads by photocleavage. In one embodiment releasing the analyte from the cells involves permeabilizing the cells in the plurality of examination areas. In one embodiment the analyte can be ribonucleic acid (RNA), e.g. messenger RNA (mRNA). In any embodiment the capture material can be a nucleotide or nucleoside sequence. In any embodiment the capture material can be a poly-T sequence and the analyte can be RNA, e.g. mRNA. Any of the methods can also involve a step of synthesizing a cDNA molecule using the RNA analyte as a template. In any embodiment identifying the analyte can involve sequencing the cDNA. In any embodiment correlating the analyte with the at least partially sequenced perturbation barcodes and spatial index barcodes can involve a step of identifying the perturbation agent and specific examination area where the cells are present.

In another aspect the invention provides methods for identifying a biological response of a cell to a perturbation agent. The methods can involve one or more of the following steps: a) dispensing cells into a plurality of examination areas present on a biochip, b) dispensing capture microbeads into the plurality of examination areas, the capture microbeads having an optical label and a capture material that binds to an analyte; c) dispensing a perturbation agent and, optionally, perturbation barcodes into the plurality of examination areas; d) releasing an analyte from the plurality of cells; e) imaging the biochip to identify the locations of the optical labels; f) binding the released analyte to the capture material on the capture microbead; g) identifying the analyte; and h) correlating the analyte with the optical imaging and spatial location of the capture bead to thereby identify a biological response of a cell to a perturbation agent.

In one embodiment the capture microbead can have the perturbation agent and optional perturbation barcodes, and dispensing the perturbation agent and the optional perturbation barcodes can involve a step of releasing the perturbation agent and optional perturbation barcodes from the capture microbead. In any embodiment the capture microbeads can not have (or can omit) a bead type identifier sequence. In any embodiment the optical label can have an emission wavelength of greater than 680 nm.

In another aspect the invention provides methods for identifying a biological response of a cell to a perturbation agent. The methods can involve one or more of the following steps: a) dispensing cells into a plurality of examination areas present on a biochip; b) dispensing perturbation microbeads into the plurality of examination areas, where the perturbation microbeads have a perturbation agent and an optical label; c) dispensing capture microbeads into the plurality of examination areas where the capture microbeads have a capture material that binds an analyte; d) imaging the biochip to identify the locations of the optical labels; e) releasing the perturbation agent from the perturbation microbeads, f) releasing an analyte from the cells; g) binding the released analyte to the capture material on the capture microbead; h) identifying the analyte; and i) correlating the analyte with the imaging to identify a biological response of a cell to a perturbation agent.

In another aspect the invention provides a capture microbead having a particle core for supporting the attachment of components; spacer-linker molecules of a first, second, and third type having proximal and distal ends, attached to the surface of the particle core at their proximal ends, and the spacer-linker molecules of the first type have a bead identifier sequence, a unique molecular identifier (UMI), and a capture material for annealing to an RNA molecule at its distal end; the spacer-linker molecules of the second type have a bead identifier sequence, a unique molecular identifier (UMI), and a first binding entity for binding to a first barcode sequence; and the spacer-linker molecules of the third type have a bead identifier sequence, a unique molecular identifier (UMI), and a second binding entity of a distinct second sequence for binding to a second barcode sequence; and where the spacer-linker molecules also have a primer binding site at their proximal and/or distal ends for synthesis of a complementary strand to a bound mRNA sequence, a bound first barcode sequence, or a bound second barcode sequence; and the bead identifier sequences on the first, second, and third types of spacer-linker molecules have the same sequence, and the unique molecular identifiers on the first, second, and third type of spacer-linker molecules each have distinct sequences, and the first and second binding entities comprise different sequences or the same sequences. In one embodiment the spacer-linker molecules, and first and second binding entities contain an oligonucleotide sequence. In another embodiment the unique molecular identifiers on the first, second, and third type of spacer-linker molecules have less than 90% sequence identity to each other.

In another aspect the invention provides methods for identifying a biological response of a cell to a perturbation agent. In any embodiment the methods involve one or more of the following steps: a) dispensing cells into a plurality of examination areas present on a biochip, where the plurality of examination areas have at least one optical label and spatial index barcodes attached to a surface in the plurality of examination areas, and the spatial index barcodes are releasable from the surface in the examination areas, b) dispensing a perturbation agent and a perturbation barcode sequence into the plurality of examination areas; c) dispensing capture microbeads into the plurality of examination areas, the capture microbeads having a capture material that binds to an analyte, a first binding entity that binds to a perturbation barcode and a second binding entity that binds to a spatial index barcode; d) imaging the biochip to identify the locations of the optical labels; e) releasing an analyte from the plurality of cells; f) releasing the spatial index barcode from the surfaces in the examination areas, g) binding the perturbation barcode and the spatial-index barcode to their respective binding elements on the capture microbead; h) binding the released analyte to the capture material; i) at least partially sequencing the bound perturbation barcode and bound spatial index barcode; j) identifying the analyte; and k) correlating the analyte, the at least partially sequenced perturbation barcode and spatial index barcode with the imaging to thereby identify a biological response of a cell to a perturbation agent. In one embodiment the perturbation barcode and the spatial index barcode are oligonucleotide sequences. In another embodiment the optical label comprises a fluorescent dye.

In another aspect the invention provides methods for identifying a biological response of a cell to a perturbation agent. In any embodiment the methods can involve one or more of the following steps: a) dispensing cells into a plurality of examination areas present on a biochip; b) dispensing a perturbation agent and a perturbation barcode into the plurality of examination areas; c) dispensing capture microbeads into the plurality of examination areas where the capture microbeads have a capture material that binds to an analyte, a binding element for binding the perturbation barcode, and an optical label; d) imaging the biochip to identify the locations of the optical labels; e) releasing an analyte from the plurality of cells; f) binding the released analyte to the capture material on the capture bead, and binding the perturbation barcode to the binding element on the capture microbead; g) identifying the analyte and the perturbation barcode; and h) correlating the analyte and the perturbation barcode with the imaging to thereby identify a biological response of a cell to a perturbation agent. In one embodiment the perturbation agent and perturbation barcode are present on perturbation microbeads, and dispensing involves releasing the perturbation agent and perturbation barcode from the perturbation microbeads.

In another aspect the invention provides a biochip for performing a molecular biology assay. In any embodiment the biochip has one or more of a) cells present in a plurality of examination areas, b) perturbation microbeads present in the plurality of examination areas where the perturbation microbeads have a perturbation agent and a perturbation barcode, c) spatial index microbeads present in the plurality of examination areas, where more than one spatial index microbead is present in a plurality of the examination areas that contain a cell, and where the spatial index microbeads have an optical label and a spatial-index barcode sequence, and the spatial-index barcode sequence is releasable from the spatial index microbeads, and d) capture microbeads present in the plurality of examination areas, the capture microbeads having a binding element that binds to the perturbation barcode and a binding element that binds to the spatial index barcode sequence, and further having a capture material that binds to an analyte. In one embodiment at least 50% of the plurality of examination areas contain at least one cell, and the biochip has at least 9,000 examination areas.

In another aspect the invention provides a cell loading device. In any embodiment the cell loading device can have one or more of the following features: a loading chip having a plurality of loading wells for loading a plurality of samples onto a biochip; a biochip having a plurality of examination areas for receiving a plurality of samples from the loading wells of the loading chip; a biochip holder for holding the biochip; a cover component; where at least one of the biochip holder or cover has at least one alignment pin that projects from the biochip holder or cover, and the other of the biochip holder or cover has at least one port for receiving and securing the at least one alignment pin; and when the alignment pins are engaged in the ports the plurality of loading wells on the loading chip are in vertical alignment with the plurality of examination areas on the biochip. In any embodiment the cover component can have at least two alignment pins, and/or the biochip holder can have at least two corresponding ports. In any embodiment the device also can have a holding component for holding the loading chip in a fixed position and/or in vertical alignment with the biochip. The device can also have at least one inlet tube that fluidly connects the exterior of the device to the biochip and that permits the flow of fluid to the loading chip, and at least one outlet tube that fluidly connects the biochip to the exterior of the biochip and permits the flow of fluid from the loading chip. The device can also have at least one inlet tube and at least one outlet tube present on the cover component. In one embodiment the biochip holder holds the biochip in a fixed position, and the holding component holds the loading chip in a fixed position. The holding component can be configured to hold the loading chip in a fixed position, and the cover can be configured to hold the holding component in a fixed position. The examination areas of the biochip can have at least 2× the volume of the loading wells of the loading chip.

### DETAILED DESCRIPTION OF THE DRAWINGS

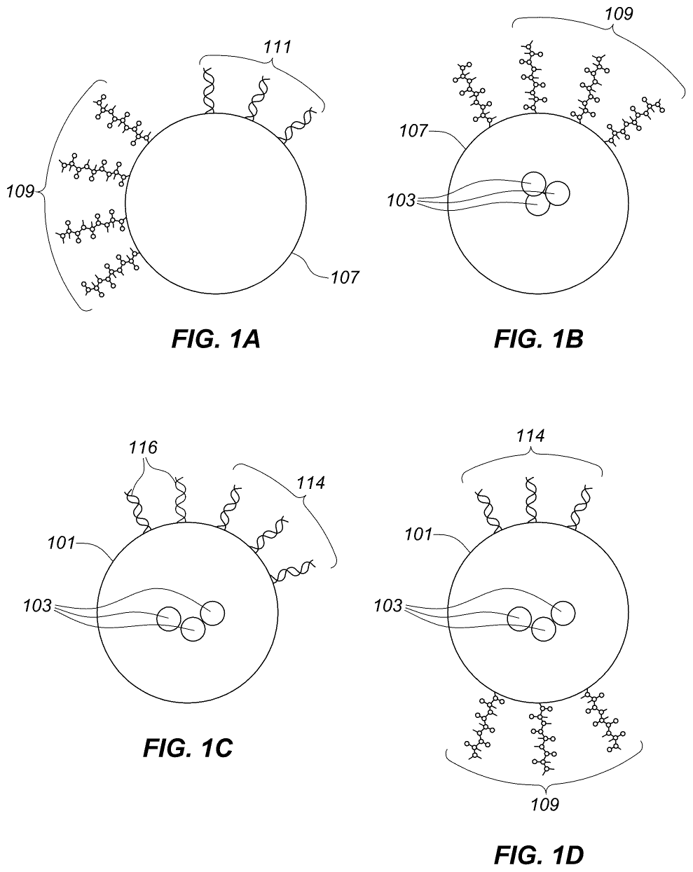

FIG. 1a provides a graphical illustration of a perturbation microbead 107 utilized in the invention. The microbead has chemical perturbation agents 109 releasably attached to its surface, and oligonucleotide perturbation barcodes 111, also releasably attached, that can be correlated to the chemical perturbation on the microbead. FIG. 1b provides a graphical illustration of a perturbation microbead 107 of the invention. In this embodiment the perturbation bead 107 has an optical label 103, which in this embodiment is depicted as fluorescent dyes or tags (and can be present on the microbead singly or in combinations), which can be detected as distinct colors and at detectable intensities. This microbead can be useful in embodiments where no spatial index microbead is utilized because the optical label is on the perturbation microbead. FIG. 1c is a graphical illustration of a capture microbead 101 of the invention. In this embodiment the capture microbead has thereon an optical label 103, capture materials 114 (e.g. a polynucleotide such as poly-(T) for capturing RNA analyte), and binding elements 116 (e.g. oligonucleotides) for capturing barcode sequences. This microbead can be useful in embodiments where no spatial index microbead is utilized because the optical label is present on the capture microbead. FIG. 1d is a graphical illustration of a capture microbead 101 of the invention. The capture microbead has an optical label 103, chemical perturbation agents 109, and capture materials 114 for capturing an analyte from a test cell. This microbead can also, optionally, have binding elements for binding perturbation barcodes, which can optionally be released and captured by the binding elements; or the perturbation on the microbead may already be known from manufacture. This microbead is useful in embodiments where it is desirable to use only a single microbead.

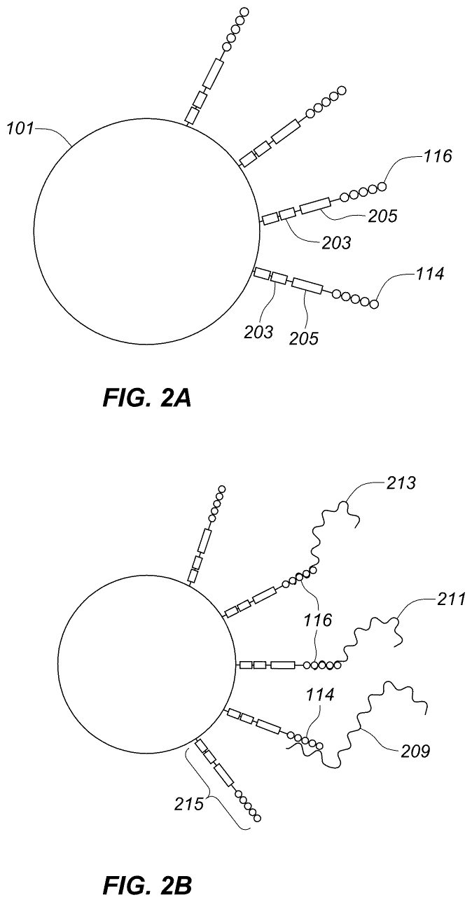

FIG. 2a is a graphical illustration of an embodiment of a capture microbead of the invention with further detail of the capture material 114 and binding elements 116. Illustrated are the capture microbead 101, and a polynucleotide having a bead ID sequence 203, a unique molecular identifier (UMI) sequence 205, and in this embodiment capture material 114 (represented as a poly(T) sequence here for RNA as the analyte) and/or a binding element 116. FIG. 2b is a graphical illustration of an embodiment of a capture microbead loaded with captured RNA analyte 209. Also depicted is captured spatial index barcode 211, and captured perturbation barcode 213.

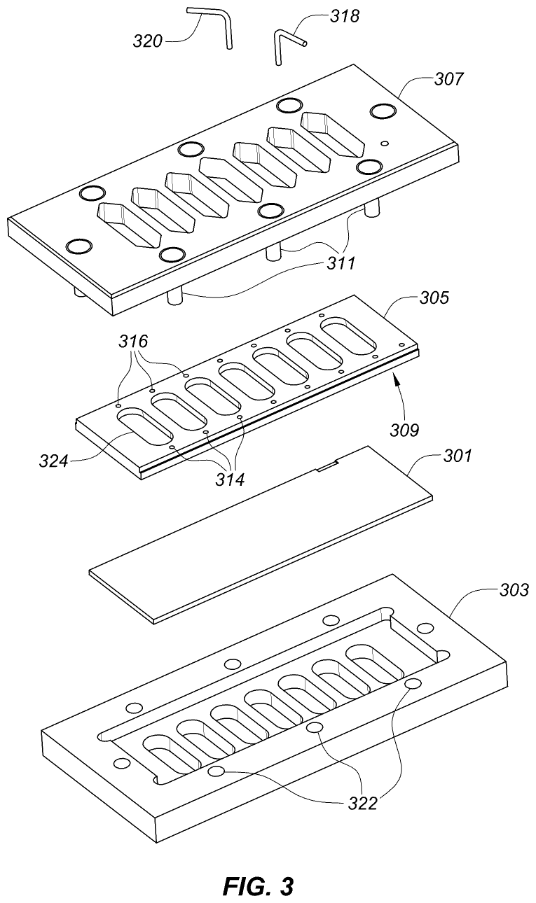

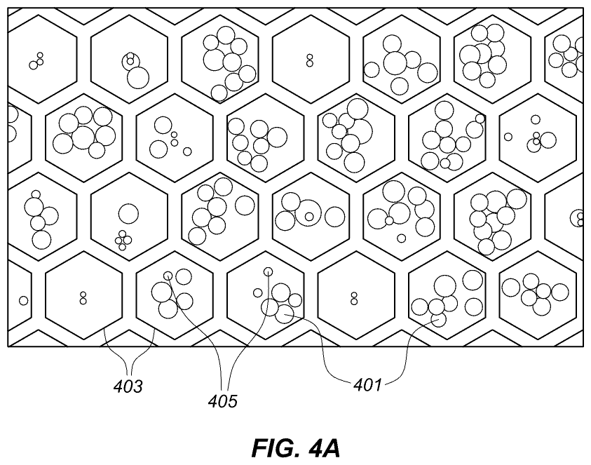

FIG. 3 provides an exploded view of a cell loading device of the invention. Depicted are a biochip 301, a biochip holder 303, a loading chip holder 305, and a cover 307 (e.g., cap) containing alignment pins 311. Not depicted is a loading chip 309 located under the loading chip holder 305. Also depicted are an inlet tube 318, outlet tube 320, inlet apertures 314 and outlet apertures 316 for distributing fluid to the biochip.

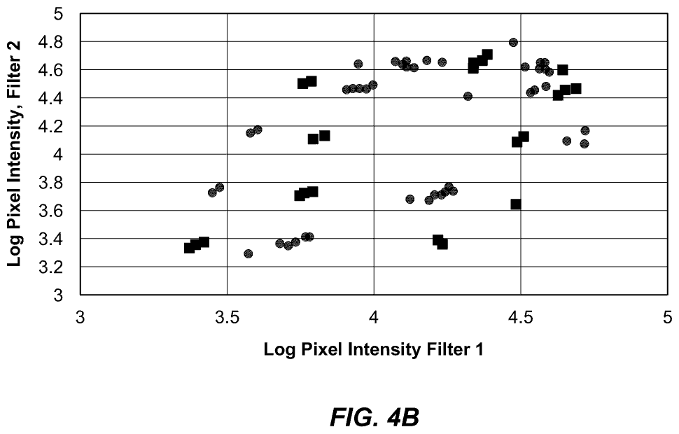

FIG. 4a provides a drawing depicting imaging data from a fluorescent microscope. In various of the examination areas 403 can be seen test cells 401, and microbeads containing an optical label 405. FIG. 4b provides a graphical illustration of data from a fluorescence microscope. The data show the colors and intensity of signals received from various optical labels contained in the microbeads.

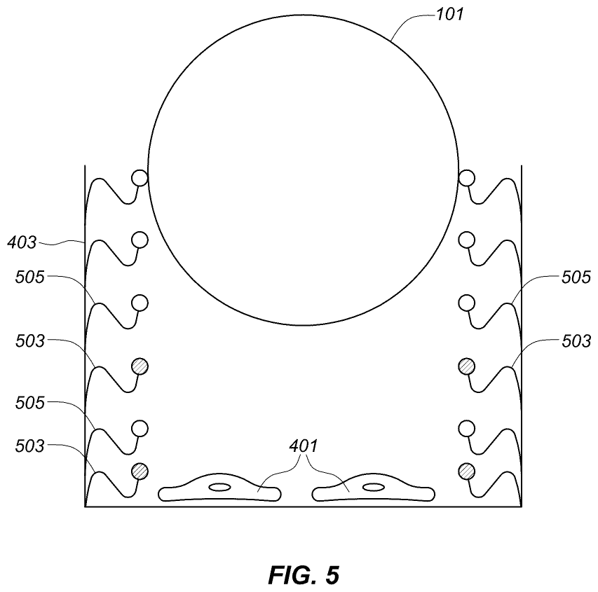

FIG. 5 provides an illustration of an examination area 403 of one embodiment of the invention having test cells 401 therein. The examination area is depicted as having a capture microbead 101, optical labels 503, and spatial index barcodes 505 present in the examination areas that identify the examination area.

### DETAILED DESCRIPTION OF THE INVENTION

The invention provides methods and compositions for ultra-high throughput and analysis of the responses of cells to a wide range of stimuli or cellular perturbations. Cellular perturbations can include anything that may elicit a response from a cell, examples including exposure to a drug candidate or small molecule, chemical, biological molecules, analytes, a particular cellular environment, and/or other stimuli. The methods permit the user to identify the response to a particular perturbation, as well as to identify the specific examination area or well on a biochip where the cell was present. Thus, the cell, the perturbation, and the cell's reaction to the perturbation can be identified. In various embodiments the stimuli or cellular perturbation can be interaction with a chemical, drug or drug candidate, antibody, biological molecule, culture condition, or an analyte.

Through the elegant use of molecular barcodes, optical labels, imaging, and nanotechnology, users are able to analyze and screen very large numbers of cells in an assay with specific cellular perturbations in short timeframes, to correlate cellular responses to the specific perturbations, as well as to retrieve a cell identical to the cell screened in the reaction for further study. The methods also provide enormous versatility in that they are able to accommodate longer cellular incubation periods necessary for some types of assays.

The invention permits the analysis or screening of very large numbers of cells that have experienced stimuli or perturbations, and the analysis of the effect of the stimuli or perturbations on the functioning of the cells.

In any embodiment the invention can involve the use of spatial index microbeads (or perturbation microbead or capture microbeads) carrying an optical label, and that do not have a unique bead type identifier sequence. The methods can therefore be performed without the need to perform sequencing of such bead type identifier sequences. This significantly simplifies the requirements not only of sequencing captured barcodes, but also of synthesizing spatial index beads used in the methods. By thus limiting the size of sequences to be amplified, the methods are designed to eliminate the need to perform extensive polymerase chain reaction (PCR) amplification steps—in the present methods the captured spatial index barcode can be amplified in a single PCR step. This is due, in part, to the use of a bead library of large size, which permits a greater distinction between individual microbeads. The methods allow such distinctions to be determined through the use of optical imaging, and thus eliminates the need to utilize microbeads (having an optical label) that carry a unique bead type indicator.

The methods can identify the reaction of a specific cell to a specific perturbation in a specific examination area (e.g. on an x-y gradient superimposed on a biochip) without interference from optical signals that are used in biological assays—and to do so in an ultra-high throughput method. This can be done, for example, by utilizing optical labels that emit at wavelengths of 680 nm or above, or 700 nm or above and leaving lower parts of the spectrum for biological assay signals. Therefore, the methods increase the accuracy of the results of both the biological assays and the spatial index analysis by decreasing interference. The methods further offer the advantage of being conducted with widely available imaging scanners, and without the need for highly specialized scanners. The methods also offer the advantage of the ability to resolve a large number of examination areas on a biochip or assay chip.

In some embodiments the invention also can utilize a bead library or bead set with a large number of unique members, which in turn permits the use of large sized examination areas in the biochip. Larger examination areas then permit the analysis of a wider variety of cell types, since some types of cells do not grow well in single cell culture, or will give a more accurate response to a perturbation agent only when in the presence of other cells, or can require a period of time to incubate to yield a valid result. The use of a large bead library or bead set (e.g. one having at least 100 or at least 200 or at least 250 or at least 400 or at least 500 members) permits a higher degree of certainty in identifying the specific microbead in an examination area since identical combinations in examination areas will be extremely rare. These larger examination areas in turn permit screening or detection assays that would otherwise not be possible or would be subject to inaccuracies. Therefore, the current methods permit a greater variety of screening assays that can be performed and a greater number of cell types that can be analyzed.

The larger volume examination areas afforded by the methods also permit multiple spatial index microbeads to be present in a single examination area, thus increasing the certainty of correctly identifying a specific microbead, for example as part of a group or configuration of microbeads in an examination area. The larger volume of the examination areas offered by the invention also decreases the chance of having two examination areas with identical spatial index microbead types after dispensing the spatial index microbeads. The methods therefore permit the examination areas to have a combination of optical labels that is unique compared to any other examination area in the plurality of examination areas on a biochip. The large volume examination areas also permit the use of larger or multiple capture microbeads, and thus greatly increase the quantity and percentage of mRNA or other analyte produced by test cells that can be collected and analyzed. The larger volume examination areas also permit the performance of assays involving cells that must be incubated with other cells or for longer periods of time while not drying out.

### Biochip

In any embodiment the methods of the invention can be performed on a biochip. The biochip can have a collection of examination areas arranged on a solid substrate that permits many tests to be performed at the same time. In various embodiments the examination areas can comprise microwells, nanowells, picowells, or microposts, or simply small structures at defined locations to which a cell or microdroplet can adhere, attach, or be immobilized. In other embodiments the examination area need not be a physical structure on the biochip, but can be a microdroplet on the biochip in which an assay can be conducted. But in other embodiments the microdroplet can be immobilized by a micropost or other structure on the biochip, and the methods can be performed inside the microdroplet.

In various embodiments the biochip can accommodate thousands of cells or biological samples. In some embodiments the biochip can contain at least 9,000 examination areas, at least 10,000, at least 25,000, or at least 35,000, or at least 50,000, or at least 70,000, or at least 100,000 examination areas. Biochips can be made of any suitable material, e.g. silicon, polydimethylsiloxane, plastic, or glass as non-limiting examples. In any embodiment during the methods the plurality of examination areas on the biochip can have a volume of 1 nl or greater, which can be loaded with at least one cell in the dispensing step. In any embodiment the examination areas can have a volume of at least 0.75 nanoliters, or at least 1 nanoliter, or at least 1.5 nanoliters, or at least 2 nanoliters, or at least 3 nanoliters, or 0.75 nl to 2 nl, or 1-2 nanoliters, or 1-2.5 nanoliters, or 1-3 nanoliters, or up to 2 nl, or up to 3 nl, or up to 4 nl, or up to 5 nl, or greater than 5 nl. In various embodiments the examination areas of the biochip can be from 100-150 um in diameter, or 100-200 um in diameter, or 150-200 um in diameter, or at least 110 um in diameter, or from 110-150 um in diameter, or about 130 um in diameter, or from 120-140 um or from 125-135 um in diameter. In various embodiments the depth of the examination areas can be about 140 um or about 150 um or about 160 um, or 130-170 um or 140-160 um or at least 120 um. The larger diameter and volume of the examination areas are advantageous since they permit more than one cell to be present in the well, as well as necessary numbers of spatial index microbeads and perturbation microbeads, as well as a capture microbead large enough to capture a maximum amount of information from the examination area. In any embodiment the method can involve loading the examination areas with one or more cells.

Some types of cells are best cultivated with one or more additional cells present in the examination area. An examination area of at least 1 nl in volume permits cultivation of more than one cell in the examination area. In any embodiment the methods can involve a step of dispensing cells into the plurality of examination areas so that more than one cell is present in the plurality of examination areas, or where at least 50% or at least 75% or at least 80% or at least 85% of the examination areas comprise more than one cell (e.g. two cells, or 3 or 4 or 5 cells).

In any embodiment the cells can be dispensed into a biochip described herein, and can be dispensed into, and analyzed in or near the examination areas such that a single cell is present in each examination area, or in at least 50% or at least 70% or at least 80% or at least 85% of the examination areas. In one embodiment the examination areas can be nanowells or microwells that can accommodate a single cell. In some embodiments the examination areas are arrays of nanowells or microwells. When the examination areas comprise microwells or nanowells, they can have any convenient shape, e.g. a flower-shaped, oval, circular, or rounded, but are not limited to any particular shape. In one embodiment the cell loading rate can be at least 80%, which can be for a single cell, more than one cell, or for microbeads of any class.

### Cell Loading Device

In some embodiments the invention utilizes a cell loading device, which permits the loading of a large number of examination areas with one or more cells. In one embodiment the cell loading device can load a single cell into a plurality of examination areas of a biochip of the invention, or into at least 50%, or at least 75% or at least 85% or at least 90% of the examination areas. With reference to FIG. 3, the cell loading device comprises a biochip 301 disposed in an upward direction onto which cells can be finally loaded. In various embodiments the biochip can have examination areas (e.g. nanowells or micro-posts) for immobilizing or trapping the cells. The biochip and examination areas can be any described herein or one otherwise suitable for use. The biochip can be secured to or within a biochip holder 303 or base component that secures and holds the biochip in a fixed or set position. The device can further have an optional holding component 305 (e.g., loading chip holder herein) that holds a loading chip 309 (not shown for visualizing the remaining components), and a cover 307 containing alignment pins 311 for vertically aligning the wells of the loading chip 309 with the examination areas of the biochip 301, with wells aligned over the examination areas when the alignment pins are engaged in the ports 322. The alignment pins 311 can project from the cover 307 and pass through or beside the optional holding component 305, and into ports 322 in the biochip holder 303 to securely hold the biochip and loading chip in appropriate orientation for the vertical alignment of the examination areas and loading chip wells and the transfer of cell samples. The alignment pins can take any shape or form, but in a preferred embodiment are pin or pillar shaped. In one embodiment the alignment pins can have a flat end, or can have an end that is otherwise shaped or configured to fit into the port, which can have complementary structures for so receiving. In various embodiments the cover or biochip holder can have at least two, or three or four or five or six or more than six alignment pins. In one embodiment the ends of the alignment pins can be star-shaped and the ports can be correspondingly star-shaped. Thus, when the alignment pins are inserted into the ports the ports can hold or otherwise fix the alignment pins in the ports, and therefore hold the loading wells and examination areas in a fixed and vertically aligned position.

In other embodiments the cover and biochip holder can have ports, and the alignment pins can be comprised on a separate component, which can be situated above the cover or below the biochip holder, or between them, and the ports can be comprised on the cover and/or biochip holder. In any embodiment the alignment pins can pass through or pass flush with the biochip holder and/or cover to secure the biochip and loading chip in fixed positions of alignment. The loading chip can be comprised of the same materials described herein for the biochip. By vertical alignment is meant that the loading chip and biochip are oriented so that a line passes through substantially the center of the loading wells and through substantially the center of the corresponding examination areas. For example, when the device is set on a flat surface the loading wells are above and vertically aligned with the examination areas cells or microbeads in the loading wells could fall by gravitational force directly into the examination areas.

Any of the methods disclosed herein can involve a step of loading a biochip utilizing a cell loading device, e.g. any disclosed herein. In use, a sample of cells can be loaded into the loading chip 309. The loading chip comprises wells that correspond to the examination areas on the biochip 301 when the alignment pins 311 are engaged, e.g. the wells on the loading chip can be oriented so that each well on the loading chip is substantially centered with respect to a corresponding examination area on the biochip. This can be accomplished through the use of the alignment pins 311 that are configured to vertically align the biochip and loading chip so that each well of the loading chip is vertically aligned with (e.g. centered within) an examination area on the biochip when the alignment pins 311 are engaged with the ports 322. The loading chip 309 can be loaded with cells, for example, by flooding the loading chip with a buffer containing cells. The wells on the loading chip can be large enough to accommodate and load only a single cell in its wells, e.g. 5-15 um or 10-15 um or 10-20 um or 15-25 um in diameter; and in various embodiments for loading cells these wells on the loading chip can be about 10 um in depth, or about 12 um in depth, or about 15 um in depth. In other embodiments for loading perturbation microbeads the wells on the loading chip can be 50-60 um, and can be about 50-60 um in depth. In general it is desirable to utilize a loading chip well of about 2 times or almost 2 times or 2-3 times the diameter of the cell or bead being loaded onto the biochip. Optionally, the examination areas on the biochip can be larger than the corresponding wells on the loading chip. The cell loading device can also have one or more inlet aperture(s) 314, into which cells and/or microbeads in a fluid can be loaded onto the loading chip. This can be accomplished by conventional flooding of the loading chip with buffer containing cells and/or microbeads, continuously until the wells are loaded. The loading buffer can exit the loading chip by one or more outlet aperture(s) 316 that permits fluid to exit the loading chip and the device. In the embodiment depicted in FIG. 3 there are shown seven windows 324, each having an inlet aperture 314 on one side and an outlet aperture 316 on the other. The device can also have an inlet tube 318, which can receive fluid and distribute it to the inlet apertures 314 for distribution to the loading chip. The device can also have outlet apertures 316, which can funnel fluid to the outlet tube 320 and allow fluid to pass to the exterior of the device.

When the loading chip 309 is loaded with cells (e.g. when at least 50% or at least 75% of the wells of the loading chip contain a cell), and the components of the device are assembled so that the alignment pins 311 are engaged in the ports 322, the well of the loading chip will be positioned directly underneath a corresponding examination area on the biochip. The loading wells can be smaller in diameter than the examination areas on the biochip. The device can then be inverted or flipped, resulting in the cells in the loading wells dropping by force of gravity into the aligned examination areas of the biochip. Thus, the examination areas will be loaded with cells. In one embodiment the examination areas are loaded with a single cell. In one embodiment the examination areas of the biochip have a larger volume than the wells of the loading chip, thus the examination areas will have room to accommodate several microbeads used in the methods, which can be loaded by using the loading chip (either simultaneously with the cells or in successive steps), or by conventional loading methods. The cell loading device therefore permits a single cell to be loaded into the examination areas of the biochip, and permits the examination areas to further accommodate additional cells, perturbation beads, spatial index beads, and capture beads for a variety of methods disclosed herein. Utilizing the cell loading device the biochip can be loaded so that at least 75% or at least 80% or at least 85% or at least 90% of the examination areas contain one or more cells-thus in various embodiments the methods have a cell loading rate of at least 75% or at least 80% or at least 85% or at least 90%.

### Cells

The methods can involve a step of dispensing a plurality of cells into a plurality of examination areas on a biochip. In one embodiment this can be done by using a cell loading device as described herein. In another embodiment the cells can be dispensed by flooding the surface of the biochip with a solution containing the cells. In other embodiments the cells can be dispensed using other methods known to persons of ordinary skill. Similarly, perturbation microbeads, spatial index microbeads, and/or capture microbeads can be dispensed into the biochip or its examination areas using the same methods.

The cells or test cells utilized in the methods of the invention can be any biological cells that the user wishes to identify the cells' biological response to a perturbation agent or other stimulus. Examples of biological cells or test cells that can be analyzed in the methods include, but are not limited to, living cells, a homogeneous or a heterogenous mixture of cells, mammalian cells, eukaryotic cells, bacterial cells, fungal cells, plant cells, prokaryotic cells, immune cells, cancer cells, antibody secreting cells, B cells, T cells, genetically modified cells, and HeLa cells. The methods can identify the biological response of a cell to a cellular perturbation. In any embodiment the cellular response can be the release of an analyte in response to the perturbation, e.g. release of an RNA molecule (e.g. mRNA), a DNA molecule, a cytokine, a polypeptide, or other response. In any embodiment the methods can involve a step of incubating a cell or cells in the examination areas for at least 8 hours, or at least 12 hours, or at least 16 hours, or at least 24 hours.

### Microbeads or Microparticles

The methods of the invention involve the use of microbeads or microparticles. Any of the microbeads or microparticles used in the methods can be of any size or shape and have any contours, and the terms “bead” or “particle” do not imply the microbeads or microparticles are necessarily spherical in shape. But in some embodiments the microbeads or microparticles can be spherical in shape. The terms microbead, microparticle, bead, and particle are used interchangeably herein.

The invention involves the use of microbeads that carry assay components. The different classifications of microbeads include perturbation microbeads, spatial index microbeads, and capture microbeads. The functional aspects of the microbeads associated with one class can sometimes be carried by a microbead of another class, and the names of the classes of microbeads are utilized only for convenience. Thus, in some embodiments the components typical of one class can be carried on a microbead of a different class, e.g. the perturbation agent and/or perturbation barcode, or the spatial index barcode can be carried on the capture microbead instead of a perturbation microbead or spatial index microbead, respectively. Similarly, in some embodiments the optical label can be carried on the capture microbead or perturbation microbead instead of the spatial index microbead. Not all methods in the invention utilize all classes of microbeads. Thus, one class of microbead in a method can carry the components normally associated with one or more microbead(s) of another class or classes. In the methods it is therefore not always necessary to have a distinct and separate perturbation microbead, spatial index microbead, and capture microbead, but rather the components and functional requirements of each can be carried by a microbead of another class.

The microbeads can be made of any material suitable for biological beads for conducting molecular biology methods known to persons of ordinary skill in the art. Examples of suitable materials for any of the categories of microbeads described herein include, but are not limited to, polystyrene (e.g. sulfonated polystyrene), polyethylene terephthalate (PET), polypropylene, poly(methyl) acrylate, poly(acrylic acid), poly(dimethyl) acrylamide, nylon, poly(lactic acid), polyoxyethylene-grafted styrene, chitosan, poly-lactic co-glycolic acid (PLGA), polyethylene glycol, and various combinations of them, as well as other materials. In any embodiment any of the microbeads can be paramagnetic, or the capture microbead can be paramagnetic. In any embodiment the microbeads can be chemically functionalized for the easy attachment or loading of perturbation agents, any kind of barcode, oligonucleotides, test compounds, molecules, or other assay components. In one embodiment the microbeads have surface carboxyl groups for functionalization.

For example, the optical label and barcodes can be present on a dedicated spatial index microbead, but such functional components can also be moved to another microbead in the methods. Non-limiting examples are illustrated in FIG. 1. FIG. 1a illustrates a perturbation microbead 107 having thereon a perturbation agent 109 and perturbation barcodes 111. In FIG. 1b there is depicted a perturbation microbead 107 having a perturbation agent 109 thereon, and also having an optical label 103 (e.g., optical code/label combination/set), here depicted as fluorescent tags attached to the microbead. Thus, in a method utilizing this microbead (or others carrying an optical label) a separate spatial index microbead may not be necessary since the perturbation microbead carries an optical code. FIG. 1c illustrates a capture microbead that also carries an optical label. And FIG. 1d illustrates a capture microbead 101 that also carries an optical label 103 and a perturbation agent 109. Thus, the components associated with one class of microbead can be present on another class of microbead, which then fulfills the functions, and provides the components of two or more classes of beads.

### Perturbation Microbeads

The invention can involve dispensing a perturbation agent and/or a perturbation barcode into a plurality of examination areas. In one embodiment the perturbation agent and/or barcode is dispensed attached to a perturbation microbead, i.e. dispensed into the examination area with the microbead. But in other embodiments the perturbation agent and/or barcode can be dispensed without being attached to or cleaved from a perturbation microbead. For example the perturbation agent and/or barcodes can be dispensed separately such as by using a cell loading device, by a Poisson distribution and flooding of the biochip containing the plurality of examination areas, or by other methods known to persons of ordinary skill.

The perturbation microbeads can comprise a perturbation agent, which is optionally releasable from the perturbation microbead. In various non-limiting embodiments the perturbation agent can be a genetic agent a chemical agent (e.g. a small molecule drug candidate or other small molecule), (e.g. a DNA or RNA molecule), a peptide (e.g. a peptide of less than 40 amino acids), a polypeptide, a protein, an enzyme, a cytokine, an antigen, an antibody, a specific binding molecule or fragment thereof, and/or a solvent, buffer, or carrier. But the perturbation agent can be any substance where it is desired to identify the biological response of a test cell to that substance. The perturbation agent (or any component attached to any of the classes of beads described herein) can be attached to the bead by a linker, which can be cleavable or releasable from the perturbation microbead (e.g. by photocleavage or other means).

Small molecules are organic compounds of low molecular weight. They can be able to easily diffuse through cell membranes to reach an intra-cellular target and have a desired therapeutic effect. In various embodiments a small molecule can be less than 500 daltons, or less than 900 daltons, or less than 1000 daltons or less than 1200 daltons or less than 1500 daltons. They can also have the ability to remain stable in the human or animal body over time, e.g. for a period of at least 3 hours, or 4 hours, or 6 hours or 24 hours or 48 hours. In one embodiment the small molecule can contain only elements selected from any one or more of carbon, nitrogen, oxygen, phosphorus, and sulfur, and all possible combinations or sub-combinations of them. Optionally, the small molecule can further comprise one or more zinc or magnesium ions.

The perturbation microbeads can also optionally contain a perturbation barcode, which in one embodiment can be an oligonucleotide sequence that identifies the perturbation agent comprised on the same microbead. In some embodiments the perturbation barcode can be a polypeptide sequence. The perturbation barcode can be unique relative to other perturbation barcodes utilized in the method, or with regard to any other barcode sequences used in the methods. In some embodiments of the methods the perturbation barcode can be bound and captured by binding elements on the capture microbead, and harvested and sequenced. The perturbation barcode captured can be correlated to the perturbation agent that was on the same perturbation microbead from which the barcode originated. Combined with the spatial index and imaging data the perturbation agent can be identified that was present in any particular examination area. When the perturbation (or spatial index) barcode is an oligonucleotide sequence, a sequence of only 10 nucleotides can provide over a million distinct barcodes, each of which can correlate to and identify a specific perturbation agent. In various embodiments when the perturbation or spatial index barcode is an oligonucleotide or polypeptide sequence the perturbation or spatial index barcode can be no more than 50 nucleotides or amino acids, or no more than 30 nucleotides or amino acids, 20 nucleotides or amino acids, or no more than 15 nucleotides or amino acids.

Total barcode sequences can include not only the perturbation or spatial index barcode, but can also include one or more of the following sequences: a head piece, a first primer sequence, one, two, three, or four barcode sequences, a second primer sequence, a UMI barcode, and a poly-T sequence. In various embodiments the total barcode sequence can be no more than 200 nucleotides in length, or no more than 150 nucleotides in length, or no more than 100 nucleotides in length, or no more than 80 nucleotides in length, or no more than 60 nucleotides in length, or no more than 50 nucleotides in length.

In some embodiments the perturbation agent and perturbation barcodes can be comprised on a microbead other than a perturbation microbead. For example, in one embodiment the perturbation agent and perturbation barcode can be comprised on a capture microbead. The capture microbead can also comprise binding elements for binding the perturbation barcodes, to thus identify the perturbation agent on the microbead. In various embodiments the perturbation microbeads and/or spatial index microbeads can be 20-60 um in diameter, or 20-50 um in diameter, or no greater than 50 um in diameter, or about 30 um in diameter, or 30-40 um in diameter, or 25-35 um in diameter, or less than 50 um in diameter, or less than 40 um in diameter, according to the needs of the particular method.

FIG. 1a depicts a perturbation microbead of the invention. The perturbation microbead 107 has perturbation agents 109, and perturbation barcodes 111, which identify the chemical perturbation on the microbead. The chemical perturbation agent 109 can be releasably attached to the microbead and released or photocleaved at an indicated time, for example with the use of ultra-violet light. FIG. 1b depicts a perturbation microbead 107 of the invention. Depicted is a chemical perturbation agent 109, releasably attached to the perturbation microbead 107, and optical labels 103. In this embodiment the perturbation agent 109 is a small molecule drug candidate. The perturbation agent 109 is releasably attached to the microbead so that it can be released at an indicated time in performance of the method and interact with the cell in the same examination area. In one embodiment the perturbation agent 109 can be attached via a photocleavable linker and cleaved by the use of light stimulus, i.e. by photocleavage (e.g. with ultra-violet light). At an appropriate time during the assay the linker is cleaved and the perturbation agent released into the assay to interact with the test cell. In this embodiment the perturbation microbeads can optionally have a perturbation barcode that identifies the perturbation agent. In another embodiment the perturbation agent can already be known from the optical label and a known perturbation agent attached to that microbead.

### Cleavable or Releasable

Any class of the microbeads or microparticles can have components attached by a linker that is cleavable or releasable from the bead, i.e. releasably attached. For example, in any embodiment the microbeads can have a linker that is a cleavable linker. In some embodiments the linker can be a photocleavable linker or a photolabile linker, for example a nitro-benzyl containing linker, or another suitable photocleavable linker such as a thermal cleavable linker, an enzyme cleavable linker, a chemical cleavable linker, a C-3 spacer arm, and/or any other suitable cleavable linkers known to persons of ordinary skill in the art. In various embodiments any of the components attached to any class of microbead can be photocleavable or photolabile, e.g. releasable by exposure to ultra-violet light, gamma rays, near red light, infra-red light, light in another part of the spectrum, enzymatic cleavage of the linker, heat cleavage of the linker.

In one embodiment the perturbation beads can have the perturbation agent and/or perturbation barcode attached by a cleavable linker, which can be a photocleavable linker. In one embodiment the spatial-index beads have spatial-index barcodes (e.g. oligonucleotides or polypeptides) attached by a cleavable linker, which can be a photocleavable linker. This enables the perturbation agent and/or spatial-index barcodes to be released at an appropriate or indicated time in the method from their respective microbeads. The perturbation agent and spatial-index barcodes can be released from the beads at the same time or at different times. Other forms of releasable attachment can also be utilized, for example release in response to a chemical stimulus or time-based release.

### Linkers

Perturbation barcodes and/or perturbation agents and/or spatial index barcodes can be attached to microbeads via linkers, for example photocleavable linkers. But the cleavable linkers utilized in the methods can be any cleavable linker. Examples include disulfide linkers, enzymatically-cleavable linkers, photocleavable linkers, diazo linkers, and photoreactive linkers. Non-limiting examples of photocleavable linkers are biotin-polyethylene glycol (PEG) 3 (or PEG4)-alkyne, biotin-PEG3-azide, biotin-PEG4-PEG3-azide, biotin-PEG3-NHS carbonate ester, biotin-PEG4, biotin-PEG4-NHS carbonate, SPDP-NHS carbonate esther, alkyne-PEG4-NHS carbonate ester, mal-NHS carbonate ester, azido-PEG3-NHS carbonate ester, azido-PEG11-NHS carbonate ester. Any of the linkers disclosed herein can be used to provide microbeads with cleavable or releasable components utilized in the methods. Persons of ordinary will realize many additional linkers and types of linkers that will find use in the invention. On any of the microbeads there can also be anchoring chemical groups that prepare the microbeads for attachment of linkers.

### Optical Labels

The methods involve the use of microbeads having optical labels, which in any embodiment can be fluorescent optical labels. In various embodiments the optical label can be carried on any one or more class of microbead, but in one embodiment is carried on a dedicated spatial index microbead. In one embodiment the fluorescent optical labels can comprise one or more fluorescent dye(s) attached to the beads as one or more optical tags, and which can be covalently or non-covalently attached. In one embodiment the dye(s) can be integrated into the composition of the microbeads to form an identifiable blended color or color code, or can be maintained as a combination of distinct dyes, or can be otherwise closely associated with the microbeads.

Examples of dyes that can be used include, but are not limited to, BP Light, BP Fluor, BP Fluor 680, BP Flour 660R, BP Fluor 647, BODIPY, Cyanine 3, cyanine 5, cyanine 5.5, cyanine 7, fluorescein, rhodamine, 800CW dye, and pyrene dyes. Other suitable dyes include any fluorescent dye having an emission wavelength of about 678 nm or greater, or about 690 nm or greater, or about 700 nm or greater, or about 720 nm or greater, or about 740 nm or greater, or about 760 nm or greater, or about 780 nm or greater.

In one embodiment the spatial index microbeads or optical labels comprise only fluorescent labels having an emission wavelength of 680 nm or greater or 700 nm or greater, or 720 nm or greater, or 740 nm or greater, or 760 nm or greater, or 780 nm or greater. This choice of optical labels has the advantage of avoiding any interference or confusion with signal that may be emitted by a label or indicator used in a biological assay also occurring in the examination area. In such an embodiment the visible spectrum can be reserved for signals produced by biological assays to avoid interference.

In some embodiments the optical label can be a dye-based optical label, e.g. a fluorescent dye, fluorescent tag, or a combination of fluorescent tags. The fluorescent dyes or tags can be attached to (e.g. covalently) or imbedded in the microbead or microparticle, or otherwise associated with the microbead. In some embodiments the optical label can be one or more fluorescent dye(s) that emit in the far red (e.g. 680-779 nm or 700-779 nm) or infra-red (780-1000 nm) portion of the spectrum. In other embodiments the optical label can be detectable in visible light or in a bright-field image. For example, the optical label can also comprise micro-structures of distinct shapes (e.g. a circular, square, triangular, or other shaped micro-structures or microfragments, etc. which can be attached to the microbeads), color ratios, and sizes. Sizes of the microbeads can also be varied to increase the size of the microbead library. In any embodiment optical labels can be detected and categorized based on both color or color code, and intensity as identifying characteristics.

In one preferred embodiment of an optical label the fluorescent dye molecules are not covalently attached to the microbead, but are integrated into the composition of the microbead and “float” within the material composition of the microbead; thus these dye molecules can be not covalently attached to the composition of the microbead, but rather are dissolved in or integrated within in. This embodiment was found to have the advantage of significantly simplifying the manufacturing process for the microbeads, enabling the manufacture of very large numbers of distinct microbeads with ease and in shorter timeframes than a conventional method of covalent attachment of the fluorescent dyes to the microbead. Thus, in one embodiment the optical label can be integrated or incorporated within the material of the microbead, but not covalently attached to the composition of the microbead, i.e. the fluorescent dyes “float” within, or very slowly diffuse within, or are dissolved within the material composition of the microbead.

Each individual microbead can be identified by its “bead signature” and quantified based on optical label signals, e.g. fluorescent reporter signals, and intensity of signal. In one embodiment the microbeads can be color-coded microspheres with two or three fluorescent dyes. Through precise concentrations of these dyes, distinctly colored bead sets of, for example, 500 5.6 μm diameter polystyrene microspheres, or 80 6.45 μm diameter magnetic microspheres can be created. Persons of ordinary skill in the art will realize that many different bead libraries can be created and utilized in the invention. The specific choices can depend on the specific application or objective of the method.

The optical label can comprise a fluorescent dye or combination of dyes, which can correspond to a color code that can be detected using an imaging device (e.g. a fluorescence scanner or other optical scanner). The color code can correspond to a specific spectral address or location on a fluorescence spectrum. The optical labels or color codes can be created so that distinct or unique spectral addresses are created by labeling beads with different ratios of fluorophores, e.g. one, two, or three, or more than three fluorophores. For example, there can be two fluorophores, one in a far red wavelength and the other an infrared wavelength and, optionally, each can be detected and distinguished by a specific color and intensity of its color. Intensity can be an identifying characteristic and can be varied through using precise concentrations of dyes. In various embodiments any combination of fluorophores or fluorescent dyes of any color combinations and/or intensities can be utilized. In some embodiments of the methods, one or more of the microbeads of the invention can be color coded. By using different colors and different intensities more than 100 or more than 250 or more than 500 spectrally distinct microbeads carrying an optical label can be designed. Adding additional characteristics as described above can expand the bead library further. The use of optical labels or color codes permits the microbeads to be correlated to a specific location or examination area on the biochip, e.g. a location on an x-y gradient superimposed on the biochip. In any embodiment an individual spatial index microbead (or other bead carrying an optical label) can be identified as part of a microbead configuration, or as part of a group of microbeads bearing an optical label within an individual examination area. The configuration can involve a specific shape indicated by the positions of the microbeads in the examination area, and can also be another identifying characteristic.

### Spatial Index Microbeads

In any embodiment the spatial index microbeads utilized in the invention can contain one or more optical labels disclosed herein having distinct optical or spectral characteristics. The spatial index microbeads can also comprise spatial index barcodes, which in various embodiments can be an oligonucleotide sequence, a polypeptide sequence, or another identifying barcode.

The spatial index barcodes can be releasable from the microbeads as described herein, e.g. can be photocleavable or can otherwise be separated from the microbeads and dispensed into the examination area. Some embodiments of the methods of the invention involve a step of releasing the spatial-index barcodes from the spatial index microbeads. The methods can involve dispensing or releasing spatial index microbeads into the plurality of examination areas, with one or more spatial index microbeads allocated to the individual examination areas; and releasing the barcodes. In any embodiment the methods can involve a step of binding the spatial index barcodes to binding elements on the capture microbeads, thus linking the captured barcode data with the imaging data. In various embodiments the spatial index barcodes can be the same length as the perturbation barcodes described herein, e.g. no more than 50 nucleotides, or no more than 30 nucleotides, or no more than 20 nucleotides or no more than 15 nucleotides. Spatial index barcodes can also be part of the total barcode sequences described herein.

In any embodiment by dispensing multiple spatial index microbeads into individual examination areas, one can determine the combination of optical labels or color codes present on the microbeads and their intensities through optical imaging of the biochip. These data can be combined with data from the sequencing of the spatial index barcodes, and therefore the specific examination area of origin for the captured analyte or bound barcodes can be determined. In other embodiments microbeads can have only one color, which can also have a measurable and specific intensity. In any embodiment there can be present in the examination areas (or in at least 50% or at least 75% or at least 85% of the examination areas) one or two or three or four or five or more than five or 3-6 or 4-8, or 5-10 or more than 10 spatial index microbeads, each having a unique and detectable optical label. In one embodiment 3-5 spatial index microbeads can be present in a plurality of examination areas, or in at least 50% or at least 75% or at least 85% of the plurality of examination areas. In any embodiment examination areas on the biochip can be identified based on the locations of the optical labels or color codes present on the spatial index microbeads. In various embodiments the spatial index microbeads can be about 5-10 um in diameter, or about 6 um in diameter or about 8 um in diameter, or about 10 um in diameter, or 5-15 um in diameter.

In any embodiment the spatial index microbeads can not comprise (or can omit or lack or exclude) a barcode or sequence that is unique to and that indicates the type of microbead, e.g. a bead type identifier (BTI). Similarly, in any embodiment the perturbation microbead or capture microbead that carries an optical label can not comprise (or can omit or lack or exclude) a barcode or sequence unique to and that indicates the type of microbead, e.g. a bead type identifier. The “type” of microbead can refer to the type of optical label on the microbead. BTI sequences can be used to identify the types of microbeads (i.e. the optical label on the microbead) used in an assay, each type having a distinct optical label. The present methods can omit such a sequence because the optical identity of a microbead can be determined with sufficient precision from the combination of optical labels imaged in a particular examination area. The absence of BTI sequences greatly eases the burden of amplifying the spatial index oligonucleotide barcodes captured in the methods. In the methods such amplification can be done with a single PCR. Since the specific identity of a spatial index microbead can be verified through the imaging data, it becomes unnecessary to produce an individualized, unique sequence attached to each spatial index microbead. The absence of BTI sequences also eases the burden of producing spatial index microbeads bearing barcodes, because the microbeads are able to use shorter barcode sequences.

The methods of the invention involve the use of a bead library (or bead set) with a large number of distinct microbeads from which the microbeads used in the methods of the invention are selected. In one embodiment the microbeads are selected from a bead library. A bead library is a set of microbeads, each having an optical label that is distinguishable (e.g. by imaging) from other microbeads in the library, and from which microbeads used in a method of the invention are selected. The microbeads in a bead library can have a fluorescent label or tag. In one embodiment all microbeads can be read in a single scan with a designated instrument, e.g. a fluorescence scanner. In one embodiment the single scan comprises detecting fluorescence emission at a single wavelength. In one embodiment the bead library comprises only microbead members having fluorescence emission wavelengths in the infra-red spectrum. In various embodiments distinctly colored microbead libraries of, for example, 500 5-6 μm in diameter polystyrene microbeads or 80 6-7 μm in diameter magnetic microbeads can be created.

In various embodiments the spatial index microbeads (or perturbation or capture microbeads carrying an optical label) are selected from a library comprising at least 100 distinct and detectable members. In other embodiments the spatial index microbeads can be selected from a library having at least 200, or at least 250, or at least 500, or at least 700, or at least 1000, distinct and detectable members. Each member of the bead library can have a distinct optical label, distinguishable from every other member of the bead library. By utilizing a large bead library the methods allow for microbeads that can each be distinguished in an examination area on a biochip by imaging, e.g. by fluorescent imaging. Imaging can localize a specific microbead (or group thereof) in a bead library to a specific examination area, as well as other microbeads in the same examination area. This ability removes the need for bead type identifier sequences, and greatly eases preparation of the microbeads in a bead library, as well as eases the harvesting of omics information from a particular microbead (e.g. a capture microbead).

### Releasing Barcodes and Analytes

The methods can involve a step of releasing the perturbation agent and/or a step of releasing perturbation barcodes from a perturbation microbead or other class of microbead carrying the perturbation agent and/or barcodes. The methods can also involve a step of releasing spatial index barcodes from spatial index microbeads. In any embodiment the perturbation agents, perturbation barcodes, spatial index barcodes, and other components can be attached to a microbead by a linker molecule. In any embodiment the methods can involve a step of releasing the perturbation barcodes and/or spatial-index barcodes from their respective microbeads and binding the barcodes to respective binding elements on the capture microbeads. In any embodiment the methods can involve a step of sequencing the captured perturbation barcodes and/or spatial index barcodes.

In other embodiments the perturbation agent and/or barcodes may not be carried on microbeads, and can be simply directly dispensed into the plurality of examination areas. Some embodiments can involve a step of dispensing a plurality of spatial index microbeads into the plurality of examination areas.

The methods can also involve a step of releasing an analyte from the plurality of cells present in the plurality of examination areas. Releasing the analyte can involve a step of permeabilizing the cell or tissue. Cells can be permeabilized by methods known to those of ordinary skill in the art, for example by lysis of the cells, by freeze-thaw protocols, exposure to materials such as solvents (e.g. acetone, methanol), detergents (Triton X-100, saponins, digitonin), exposure to calcium chloride, mechanical disruption, liquid homogenization, sonication, as well as other methods. The specific method used to permeabilize the test cell can depend on the analyte sought to be studied. Persons of ordinary skill in the art will realize appropriate permeabilization methods for a particular application. In various embodiments the analyte can be mRNA, a cytokine, antigens, aptamers, antibodies, or any analyte that may be produced by the test cell in response to a perturbation.

The methods can also involve a step of identifying the analyte. When the analyte is mRNA, other RNA, or DNA, the analyte can be identified by sequencing the analyte. When the analyte is a cytokine it can be identified by an enzyme-linked immunosorbent assay (ELISA) assay, nuclear magnetic resonance (NMR), or another method in an assay step.

### Capture Bead

In the methods the capture microbeads can have binding elements (e.g. oligonucleotide sequences) that can bind to (or are complementary to) the perturbation barcodes and/or spatial index barcodes (which in one embodiment can also be oligonucleotides) released or dispensed in the methods. In one embodiment the binding elements contain poly(T) sequences, and any one or more of the spatial index barcodes, perturbation barcodes (e.g. oligonucleotides), and RNA released by the test cell can have complementary poly(A) sequences. But the binding elements can be any sequence that binds to a complementary sequence on the perturbation barcode and/or spatial index barcode in the same examination area. In any embodiment more than one capture microbead can be dispensed into the plurality of examination areas. For example, there can be dispensed 2 or at least 2, 3 or at least 3, 4 or at least 4, 5 or at least 5, or between 3 and 5, or 6 or at least 6, or more than 6 capture microbeads into at least 50% or at least 75% of the plurality of the examination areas on the biochip.

The capture microbead can also have a capture material that binds to an analyte produced or secreted by a cell. In one embodiment where the analyte is RNA (e.g. mRNA) the capture material can be, for example, a poly-T sequence that can bind and immobilize the poly-A tail of RNA produced by the cell. But any suitable capture material that binds to and captures the analyte, for example a DNA or RNA sequence, an antibody or portion of an antibody, can be utilized depending on the analyte being studied. In one embodiment the methods can further involve a step of synthesizing a cDNA molecule using the captured RNA analyte as a template and, optionally, sequencing the cDNA. Many proteins or polypeptides bind to a DNA or RNA sequence or an antibody or receptor, or portion thereof. In one embodiment the analyte is a cytokine, and the capture material can be a cytokine receptor or portion thereof. In other embodiments the capture material can be any specific binding substance, e.g. biotin, avidin, streptavidin, a portion of an antibody binding domain, a receptor or a portion of a receptor that binds an analyte, an aptamer, or any specific binding molecule suitable for the particular application. In any embodiment the methods can involve a step of binding an analyte released or secreted by a cell to a capture material on the capture microbead.

The methods can also involve a step of binding a released perturbation barcode and/or a released spatial index barcode to respective binding elements on a capture microbead. In one embodiment the capture microbead can therefore have bound to it a perturbation barcode and/or a spatial index barcode, and the analyte (e.g. mRNA bound to a poly-T sequence on the capture bead) bound to a capture material. The captured perturbation barcode can reveal the specific perturbation agent the cell was exposed to. The captured RNA can reveal the biological response of the cell being tested. And the captured spatial index barcode can identify the specific examination area where the reaction occurred when combined with imaging data. This data can also be correlated with the type of cell that was present in the examination area.

In various embodiments the capture microbead can have capture groups 215 attached to it that provide information about the bead or components attached to it. In one embodiment the capture groups comprise a series of nucleic acid sequences (e.g. barcodes), which can be a consecutive series. Thus the capture groups on the capture microbead can have a bead ID sequence (BID) 203, a unique molecular identifier 205, and a poly(T) or other analyte capture material 114 and/or binding element 116. The capture groups can also have one or more primer sequences thereon. The bead ID can comprise a barcode sequence that uniquely identifies the specific bead from which it came. In one embodiment the bead ID can indicate the captured analyte or barcode sequence came from a microbead having a specific spectral address, resulting from the combinations of dyes and/or their intensities used in its synthesis. Thus, for example, if a bead library has 500 beads, then 500 bead ID sequences can be utilized, a unique sequence on each microbead. Each capture group for a specific bead can have the same bead ID sequence. This can be important in identifying the specific microbead that a captured barcode or analyte originated from because each captured barcode or analyte can be harvested with a bead ID sequence on it. In one embodiment the BID can be an oligonucleotide sequence specific to the individual bead.

The capture groups of the microbead can also have a unique molecular identifier (UMI) sequence. Each UMI on any individual microbead is a unique sequence. Thus, each microbead can have many UMIs on it, and each UMI on any individual bead is different from any other UMI on the same bead. The UMI can be useful for quantifying the result of the perturbation on a test cell, since copies having the same bead ID and same UMI can be eliminated as mere copies produced during amplification. The capture group can be harvested from the capture microbead, and can provide the bead ID, the UMI, and the captured analyte or barcode. Since each UMI on a microbead is distinct, the number of UMIs that have an associated captured analyte permits the quantitation of the captured analyte, as each unique captured analyte will be associated with a unique UMI. Thus, upon sequencing of the capture groups it is shown that each UMI sequence is attached to a distinct harvested analyte. Duplicate sequences can be subtracted from the quantitation total.

The capture microbeads can be of any suitable size and shape. Advantageously the capture microbeads can be generally spherical in shape. The capture microbeads are also advantageously larger in size in order to accommodate the maximum number of analytes and barcodes produced by a test cell. In various embodiments the capture beads are from 70-110 um or from 70-100 um in diameter, or 70 um or greater in diameter, or from 70-80 um in diameter, or 75 um or greater in diameter, or from 75-100 um or from 80-100 um or from 85-95 um or about 90 um in diameter, or from 80-110 um in diameter.

In various embodiments the capture microbeads can capture 10,000-25,000 mRNA molecules per cell on its capture material, or from 25,000-50,000 mRNA molecules per cell, or from 50,000-75,000 mRNA molecules per cell, or greater than 75,000 mRNA molecules per cell. In one embodiment the capture microbeads are about 90 um in diameter and can capture at least 10,000 mRNA per cell or from 10,000 to about 25,000 mRNA molecules per cell.

FIG. 1c depicts a capture microbead 101 that has optical labels 103. The microbead also has capture material 114 (e.g. poly(T) for binding to a poly(A) tail of mRNA) for capturing analytes produced by a test cell, and binding elements 116 for binding perturbation barcodes. In this embodiment of the methods no spatial index barcodes are needed to conduct the assay as the optical label is on the capture microbead. This capture microbead can thus be useful in a method that does not utilize a spatial index microbead.

FIG. 1d illustrates a capture microbead 101 that has capture material 114, an optical label 103, and a perturbation agent 109. This microbead can be useful in a single microbead method, because the capture microbead itself provides the optical label, perturbation agent, capture material, and binding elements. This capture microbead can optionally also have perturbation barcodes that identify the perturbation agent on the microbead. In other embodiments the perturbation agent can already be known by identity of the microbead through the optical label and knowledge of the perturbation agent attached during synthesis.

FIG. 2a depicts a capture microbead 101. The capture microbead has an attached bead ID sequence (BID) 203, and can also have a unique molecular identifier (UMI) 205. In the embodiment depicted the microbead has a capture material 114 (e.g. a poly(T) sequence to capture mRNA). The microbead can also have a binding element 116 for binding of spatial index and/or perturbation barcodes. The bead ID, UMI, and capture material or binding element can be present consecutively, as illustrated here. In other embodiment the positions of the bead ID and UMI can be reversed, or can be present in another sequence.

FIG. 2b illustrates a capture microbead providing a more detailed view of the capture group 215. The capture group can contain a capture material 114, here depicted with bound RNA analyte 209. Also shown are binding elements 116 depicted with bound spatial index barcode oligonucleotide 211. Additional binding elements 116 are shown with bound perturbation barcode oligonucleotides 213. In any embodiment amplification of the capture group and analyte or barcode bound thereto can result in a DNA molecule that can identify the capture microbead, a UMI for quantification, the perturbation barcode and/or spatial index barcode, as well as identify and quantify the bound analyte.

### Imaging

The methods can involve a step of imaging the biochip to identify specific spatial index microbeads and the precise examination areas where they are located. Imaging of the biochip permits the examination areas to be distinguished, and imaging can detect the spectral address or color code or other optical label, combined with the physical location of each optically coded microbead. When the optical label is a fluorescent dye or tag (or combination thereof) associated with the microbeads, the imaging can be fluorescence imaging. In some embodiments the imaging can be done with a fluorescence microscope, which can be equipped with a narrow bandpass filter. Persons of ordinary skill in the art know how to select suitable bandpass filters for a particular application where fluorescent dyes or molecules are used. Various imaging methods are known to persons of ordinary skill in the art. In various embodiments the imaging can be fluorescence imaging, or fluorescent flow cytometry, or quantitative fluorescent microscopy. In various embodiments the images can be produced from microscopy, imaging probes, spectroscopy, a combination of any of them, or by other means known to persons of ordinary skill in the art. In one embodiment the imaging can be performed by fluorescent excitation at a single wavelength.

Each individual optically labeled microbead can be identified by its spectral or “bead signature,” and therefore correlated to a specific examination area on an x-y gradient superimposed over the biochip, with each examination area representing a specific x-y point. The sequences yielded by an individual bead can be identified based on the sequences harvested. And the results of the analysis can be quantified based on the optical labels and sequences harvested from the individual microbead. By imaging the bead signatures or color codes or other optical label, the specific examination area that was the origin of the optical labels can be identified from the imaging, and therefore also the barcodes and captured analyte in the examination area can be determined. Thus, the cell from which the analyte came and the perturbation agent can thus also be determined. The methods therefore permit ultra-high throughput analysis of the biological responses of cells to specific perturbation agents.

In some embodiments it is not necessary that functions be allocated to separate or specific microbeads. Thus, any of the microbeads can carry one or more than one of the functions described, as illustrated in FIGS. 1a-d. For example, the capture microbead can also carry the optical label in addition to capture materials for the analytes, the perturbation barcodes (when used), and binding entities for capturing barcodes. Or the perturbation microbead can carry the optical label. Thus, in considering the invention one microbead may fulfill more than the named function that might otherwise be fulfilled by another bead class. Thus, the capture microbead can also perform the functions of the spatial index microbead or perturbation microbead. Likewise, the spatial index microbead can also perform the functions of the capture microbead and/or the perturbation microbead. With reference to the present disclosure persons of ordinary skill will be able to make design choices that meet the requirements of any particular application of the methods.

In any of the embodiments the capture microbeads can also be magnetic, and thus facilitate harvesting of the beads by magnetism and sequencing of captured barcodes and/or analyte.

### Correlating

The methods of the invention can also involve a step of at least partially sequencing the perturbation barcodes and/or the spatial index barcodes bound to a binding element on the capture microbead, or complement sequences of either or both. In one embodiment the perturbation barcodes and/or spatial index barcodes can be oligonucleotides. In other embodiments the perturbation barcodes and/or spatial index barcodes can be a polypeptide sequence.

The methods of the invention can involve a step of correlating the analyte bound to the capture material of the capture microbead with the optical imaging and perturbation barcode to identify the biological response of a cell to a perturbation agent. The methods can also involve a step of correlating the perturbation barcodes and/or spatial index barcodes bound to the binding elements on the same capture microbead that bound the captured analyte with the optical imaging, and thereby identify the bead of origin for these sequence(s). This permits correlation of the analyte and/or spatial index barcodes and/or perturbation barcodes with a specific microbead identified in the optical imaging and specific perturbation agent. Thus, the specific examination area containing the individual capture microbead can be identified on an x-y gradient superimposed over the biochip. The perturbation barcode harvested from the same capture microbead identifies the perturbation agent used in the assay in the identified examination area. The analyte and spatial index barcode (when utilized) (and perturbation barcode) also correlate to the capture microbead via the bead ID sequence. The quantity of the analyte can also be calculated using the UMI sequences. Therefore, by correlating these data the methods allow the identification of the specific examination area where the method was conducted, the perturbation agent present, the identity of the cell in the examination area (e.g. using phenotypic data), and the analyte produced by the cell in response to the perturbation agent.

### Methods

In any embodiment the method steps disclosed herein do not necessarily have to be performed in the named order. For example, the capture microbeads can be introduced prior to or after releasing the perturbation agents and perturbation barcodes from the perturbation microbeads, or before or after introducing the spatial index microbeads. Similarly, other steps can also be performed in alternative order.

In various embodiments the methods can probe or test at least 5,000 examination areas, or at least 9,000 examination areas or cells, or at least 10,000 examination areas or cells or at least 25,000 examination areas or cells, or at least 35,000 examination areas or cells, or at least 50,000 examination areas or cells, all simultaneously.

The methods disclosed herein also permit the detection and quantitation of multiple analytes at the same time, by using multiple capture moieties on the capture microbeads.

Any of the methods described above can also involve any one or more of the steps of: optionally imaging the biochip to identify the locations of the spatial index beads or optical labels; optionally releasing the perturbation agent and/or perturbation barcodes from the perturbation microbeads or capture microbeads; optionally releasing an analyte from the plurality of cells; optionally releasing the spatial-index oligonucleotide sequences from the plurality of spatial index beads; optionally binding the released perturbation barcode and/or the released spatial-index oligonucleotide barcodes to the binding elements of the capture beads; optionally binding the released analyte to the capture material on a capture microbead; optionally at least partially sequencing the annealed perturbation barcodes and annealed spatial-index oligonucleotides or their complement sequences; and identifying the analyte and/or the perturbation barcode; and correlating the analyte, and/or the at least partially sequenced perturbation barcodes and/or spatial index oligonucleotides with the optical imaging to thereby identify a biological response of a cell to a perturbation agent.

### Example 1

Forty-eight carboxylated polystyrene magnetic microspheres impregnated with three spectrally distinct fluorescent dyes were selected for use as spatial index microbeads in this procedure. Oligonucleotides containing a photocleavable group were grafted onto the microbeads via ethylene dichloride (EDC)/sulfo-NHS coupling. Barcoded poly-A sequences were then extended onto the SI beads via extension with DNA polymerase. Double-stranded DNA was then denatured with NaOH, leaving a single-strand of barcoded dA sequences on the bead. Final barcoded beads were determined to have approximately 100,000 oligos/bead.

The assay biochip was conjoined with a loading biochip using a cell loading device. The two biochips were aligned so that the transfer chip wells were vertically aligned with the examination areas on the biochip. In this embodiment capture-compound microbeads were used, where the capture microbeads also contained the perturbation compound for testing the cells as well as perturbation barcodes identifying the compound on the microbeads. The capture-compound microbeads were thus loaded onto the transfer chip and then the chip flipped so that the beads fell into the assay chip wells. The capture-compound beads contained small molecule perturbation agents (compounds) from a library of about 750,000 members linked via a photocleavable linker. The capture-compound microbeads also contained non-photocleavable DNA encoded capture material containing DNA linker, a primer binding site, compound-specific (perturbation) barcode sequences, binding elements for capturing spatial index barcodes, a unique molecular identifier (UMI) sequence, and a dT30 sequence.

The biochip was washed with TE/TW (0.02% or 0.1% Tween in Tris-EDTA (TE) buffer) buffer and about 500,000 washed capture-compound microbeads were loaded onto the biochip. The biochip was inspected to ensure that the capture microbeads were evenly loaded. Once about 99% of the transfer wells were occupied by a single microbead, excess microbeads were quickly removed from the biochip using TE/TW buffer and recovered for future chip loadings.

Approximately 350,000 spatial-index microbeads were washed 3× on a magnet using TE/TW buffer and then loaded onto the loading chip in TE/TW buffer. Once the microbeads were evenly distributed across the wells of the loading chip, the chip was flipped so that approximately five spatial-index microbeads fell into each examination area. The examination areas thus contained a single capture-compound microbead, and approximately 5 spatial-index microbeads. Excess spatial-index beads were then washed off the chip.

After loading the biochip was imaged using brightfield imaging to record the location of the spatial index microbeads (FIG. 4a) as well as the intensity levels (FIG. 4b) of the three fluorescent dyes in the beads. The biochip was washed 3× with a warmed cell culture media (10% fetal bovine serum (FBS) and penicillin G and streptomycin (pen/strep)), and about 1.5 million immortal human T-lymphocyte cells (Jurkat) were loaded onto the biochip as described above so that about 20 cells were loaded in each well. Cells were allowed to settle for 5-10 minutes before additional washing. About 2 ml of liquid fluorinated oil (FC-40) was loaded onto the biochip for oil capping.

The biochip was exposed to uv light to release the perturbation agent using 10 mW/cm2 365 nm for one minute. The biochip was then incubated 18 h at 5% in a CO2 incubator at 37° C. The biochip was then imaged in brightfield and green fluorescent protein (GFP) channels to measure the intensity of the GFP signal from the cells. After imaging, the biochip was exposed to 20 mW/cm2 at 365 nm for 5 additional minutes to ensure all spatial index barcodes were released from the spatial index microbeads. After UV exposure the biochip was placed at −80° C. overnight, or for at least 20 minutes if processed immediately.

Two freeze-thaw lysis cycles of the cells were performed for at least 15 minutes at 80° C. and 15 minutes of thawing. After the final thaw, an additional 30 minutes at room temperature was included to allow for complete hybridization of RNA and spatial index barcodes to the bead. FT-TW buffer (1 M NaCl, 100 mM Tris-HCl pH 8.0, 10 mM ethylenediaminetetraacetic acid (EDTA), 0.1% Tween (TW), 5 mM dithiothreitol (DTT)) was slowly loaded over the biochip. Microbeads dislodge by the FT-TW buffer flow were collected into a centrifuge tube containing 1 ml of FT-TW buffer. When all microbeads had been collected the collection tube was spun down and the supernatant removed. After pelleting the capture microbeads were transferred into a clean tube and given additional washes in FT-TW buffer.

The bead pellet was resuspended in 300 uL of reverse transcriptase (RT) MM (1× RT buffer, 1 mM dNTP, 12% PEG 8000, 0.25 U/uL Protector RNAse inhibitor, 15 U/uL of Maxim H-reverse transcriptase). The resuspended microbeads were then incubated 15 minutes at 37° C. with end-over-end rotation for 15 minutes and then 45 minutes at 50° C. with end-over-end rotation. After, the microbeads were pelted with 1000×G centrifugation for 1 minute and then resuspended in 1 mL TE/SDS (0.1% sodium dodecyl sulfate (SDS) in TE buffer) buffer and then centrifuged at 1000×G for 1 minute. The bead pellet was then washed twice with 1 mL of TE/TW (0.02%) buffer. Afterwards, the microbeads were subjected to centrifugation at 1000×G and washed with TE/TW (0.02%) buffer.

The capture-compound microbeads were again pelleted and resuspended in 1× Exonuclease 1 master mix and then incubated at 37° C. for 30 minutes with end-over-end rotation. After the compound-capture beads were pelleted and resuspended in 1 ml of TE/SDS (0.1%) and twice in 1 ml TE/TW (0.02%).

The capture-compound microbeads were pelleted and resuspended in 120 ul of 0.1 N NaOH and mixed, then incubated at room temperature for 5 minutes with end-over-end rotation. 120 um of 200 mM Tris-HCl, pH 8.0 was added to the denatured microbeads and spun down at 1000×G for 1 minute. 240 ul of the supernatant was transferred into a new microcentrifuge tube for synthesis, and a 2.5× solid-phase reversible immobilization (SPRI) bead cleanup performed by adding 600 ul of SPRI beads to the supernatant product. The SPRI beads were split into 4 PCR strip tubes (˜210 uL) and incubated 5 minutes, then washed 2× on a magnet with 80% ethanol. The DNA from the first tube was eluted in 45 uL low TE buffer and then that same 45 uL was used to elute the DNA from the beads in tubes 2-4.

The 45 uL eluent was used to amplify and add P5 and P7 adapters using DNA polymerase MM. A 1.2×SPRI bead clean-up was performed twice by first adding 120 uL of SPRI beads to the PCR MM and incubating 5 minutes at RT. Two washes with 200 uL of 80% ethanol were performed, and the final product quantitated with a fluorometer and then a PCR electrophoresis gel ran to ensure the final product was of the expected length.

The denatured capture microbeads were further washed with Exonuclease 1 and then further washed by resuspending the bead pellet in 1 mL of TE/TW (0.02%) and centrifuging 1000×G for 1 minute and then resuspending the bead pellet in 1 mL of water and centrifuging 1000×G for 1 minute. The capture microbeads were resuspended in 300 uL of 2^{nd} Strand synthesis MM (1×NEB Buffer 2 (New England Biolabs), 12% PEG, 1 mM dNTP, 10 uM Randomer-oligo, 0.125 U/uL Klenow (Exo-)). The capture microbeads were then incubated at 37° C. for 30 minutes with end-over-end rotation. After, the beads were centrifuged at 1000×G for 1 minute and resuspended with 1 mL of TE/SDS (0.1%). The bead pellet was then washed with 1 mL of TE/TW (0.02%) and centrifuged at 1000×G for 1 minute, and again with water.

The bead pellet was then resuspended in 120 uL of 0.1 N NaOH and incubated for 5 min at room temperature with end-over-end rotation, then neutralized by adding 120 uL of 200 mM Tris-HCl pH 8.0, and centrifuged at 1000×G for 1 minute. About 240 uL of the supernatant was transferred to a new microcentrifuge tube. A 1.6×SPRI bead cleanup was performed by adding 400 uL of SPRI beads to the neutralized denatured product and incubated for 5 minutes at room temperature. The microbeads were then split into 3 PCR strip tubes and the supernatant removed. Two washes with 80% ethanol were performed and then the first tube eluted in 45 uL of low TE, and the same 45 uL was then used to elute the 2^{nd} and 3^{rd} tubes.

Whole transcriptome amplification (WTA) was then performed from the eluted product using primers specific to the bead side of the captured cDNA product and the other specific to the randomer-oligo. Several cycles of PCR were performed using the following PCR program (initial denature at 98° C. for 1 minute, 7 cycles of 98° C. for 20 seconds, 30 seconds at 58° C., 2 minutes 72° C., and a final extension step of 72° C. for 2 minutes. A 1.0×SPRI bead clean-up was performed with SPRI beads on the PCR product. The beads were incubated for 5 minutes and then placed on a magnet to remove the supernatant, and an 80% ethanol wash performed on the magnet. Beads were eluted in 45 uL of low TE buffer.

The eluted PCR product was then amplified to add indexed cluster formation primer sites (P7 and P5). Six cycles of PCR were performed using the following PCR program (Initial denature at 98° C. for 1 minute, 7 cycles of 98° C. for 20 seconds, 30 seconds at 58° C., 2 minutes 72° C., and a final extension step of 72° C. for 2 minutes). After, a double-sided SPRI bead cleanup was performed by first adding 55 uL to 100 uL of PCR product and then incubated 5 min at RT. The product was then placed on the magnet and the supernatant was transferred to a new PCR strip tube containing 25 uL of SPRI beads and incubated for 5 minutes at room temperature. The beads were then placed on a magnet and the supernatant removed. Two washes of 200 uL of 80% ethanol were performed on the magnet and then the beads eluted in 33 uL of low TE buffer.

### Quantification and Sequencing

The final library was then quantitated using a fluorometer and DNA gel electrophoresis was performed for verification, and the DNA was found to be of the expected size (350-700 bp).

The mRNA and spatial-index barcode libraries were sequenced on a commercially available sequencer using standard primers provided by the commercial vendor with a flow-cell at a 1:8 ratio (Spatial: mRNA). 800 pM was loaded onto the flow cell. Standard, on-board sequencing primers provided by the manufacturer were used, where Read 1 sequences the cDNA read for the mRNA libraries and the spatial-index barcode for the spatial-index libraries. Read 1 used the manufacturer-recommended Read 2 sequencing primer binding site and sequenced at 68 bp. Index 1 sequenced the perturbation barcode and used the manufacturer-provided Index 1 sequencing primer binding site and sequenced at 43 bp. Index 2 sequenced the sample index primer and used the manufacturer provided Index 1 primer binding site and sequenced at 8 bp. Read 2 sequenced the unique molecular identifier (UMI) and used the manufacturer provided Read sequencing prime binding site and sequenced at 14 bp.

### EMBODIMENTS

Each of the following embodiments are representative of a portion of the possible combinations that can be achieved herein, many of which are explicitly provided above.

Embodiment 1. A method for identifying a biological response of a cell to a perturbation agent, comprising,
a) dispensing cells into a plurality of examination areas comprised on a biochip,
b) dispensing a perturbation agent and a perturbation barcode into the plurality of examination areas,
c) dispensing spatial index microbeads into the plurality of examination areas, wherein the spatial index microbeads comprise optical labels and spatial-index barcodes that are releasable from the spatial index microbeads, and the spatial index microbeads do not comprise a bead type identifier sequence;
d) dispensing capture microbeads into the plurality of examination areas, the capture microbeads comprising binding elements for binding the perturbation barcodes, and binding elements for binding the spatial-index barcodes, and further comprising a capture material that binds to an analyte produced by the cells;
e) imaging the biochip to identify the locations of the spatial index microbeads;
f) releasing an analyte from the plurality of cells;
g) releasing the spatial-index barcodes from the spatial index microbeads,
h) binding the released perturbation barcodes and the released spatial-index barcodes to the respective binding elements on the capture microbeads for binding the perturbation barcodes and spatial index barcodes,
i) binding the released analyte to the capture material on the capture microbead;
j) at least partially sequencing the bound perturbation barcodes and the bound spatial-index barcodes, or their complement sequences;
k) identifying the analyte; and
l) correlating the analyte, the at least partially sequenced perturbation barcodes and spatial index barcodes, or their complement sequences, with the optical imaging to thereby identify a biological response of a cell to a perturbation agent.

Embodiment 2. The method of embodiment 1 wherein the perturbation agent and perturbation barcodes are comprised on the capture microbeads or on perturbation microbeads, and the perturbation barcodes comprise an oligonucleotide sequence, and the perturbation agent and barcodes are dispensed by releasing them from the capture microbeads or perturbation microbeads.

Embodiment 3. The method of embodiment 1 wherein the perturbation agent and perturbation barcodes are comprised on perturbation microbeads, and the perturbation barcodes comprise an oligonucleotide sequence, and the perturbation agent and barcodes are dispensed by releasing them from the perturbation microbeads.

Embodiment 4. The method of any one of embodiments 1-3 wherein the spatial index barcodes comprise oligonucleotide sequences.

Embodiment 5. The method of any one of embodiments 1-4 wherein the biochip comprises at least 9,000 examination areas having a volume of 1 nanoliter or greater.

Embodiment 6. The method of any one of embodiments 1-5 wherein the cells are dispensed so that at least 50% of the plurality of examination areas contain a single cell.

Embodiment 7. The method of any one of embodiments 1-6 wherein the spatial index microbeads do not comprise a bead type identifier (BID) sequence.

Embodiment 8. The method of any one of embodiments 1-7 wherein the method does not involve the sequencing of a bead type identifier sequence.

Embodiment 9. The method of any one of embodiments 1-8 further comprising incubating the cells in the examination areas for at least 8 hours.

Embodiment 10. The method of any one of embodiments 1-9 wherein the spatial index microbeads are selected from a bead library of at least 100 or 250 beads that each have a distinct optical label.

Embodiment 11. The method of any one of embodiments 1-10 wherein the spatial index microbeads comprise fluorescent dyes having an emission peak at a wavelength of 680 um or greater.

Embodiment 12. The method of any one of embodiments 1-11 wherein at least 50% of the examination areas have more than one cell from the plurality of cells.

Embodiment 13. The method of any one of embodiments 1-12 wherein the spatial index microbeads in a well comprise a combination of optical labels that is unique relative to any other examination area in the plurality of examination areas.

Embodiment 14. The method of any one of embodiments 1-13 wherein the spatial index beads are chosen from a bead library comprising at least 100 distinct beads.

Embodiment 15. The method of any one of embodiments 1-14 wherein the spatial index barcodes comprise an oligonucleotide sequence of no more than 150 nucleotides.

Embodiment 16. The method of any one of embodiments 1-15 wherein the spatial index barcodes are oligonucleotides and the binding elements and capture material are comprised on a capture group that comprises a unique molecular identifier sequence.

Embodiment 17. The method of any one of embodiments 1-16 wherein the optical label comprises a fluorescent dye that comprises a color code.

Embodiment 18. The method of any one of embodiments 1-17 wherein the examination areas on the biochip are identified based on the locations of the color codes from the spatial index microbeads.

Embodiment 19. The method of any one of embodiments 1-18 wherein the color codes are correlated to a specific examination area on an x-y gradient on the biochip.

Embodiment 20. The method of any one of embodiments 1-19 wherein more than one spatial index microbead is present in at least 50% of the examination areas.

Embodiment 21. The method of any one of embodiments 1-20 comprising dispensing at least three capture microbeads into a majority of the examination areas.

Embodiment 22. The method of any one of embodiments 1-21 wherein the examination areas are wells at least 110 um in diameter.

Embodiment 23. The method of any one of embodiments 1-22 wherein the spatial index beads and perturbation beads are 20 50 um in diameter.

Embodiment 24. The method of any one of embodiments 1-23 wherein the capture beads are at least 75 um in diameter.

Embodiment 25. The method of any one of embodiments 1-24 comprising

dispensing more than one cell to each examination area.

Embodiment 26. The method of any one of embodiments 1-25 wherein the perturbation agent is a small molecule drug candidate.

Embodiment 27. The method of any one of embodiments 1-26 wherein the perturbation agent comprises a nucleic acid sequence.

Embodiment 28. The method of any one of embodiments 3-27 wherein the perturbation agent and the perturbation barcode are released from the perturbation microbead by photocleavage.

Embodiment 29. The method of any one of embodiments 1-28 wherein the spatial-index barcodes are released from the spatial index microbeads by photocleavage.

Embodiment 30. The method of any one of embodiments 1-29 wherein releasing the analyte from the cells comprises permeabilizing the cells in the plurality of examination areas.

Embodiment 31. The method of any one of embodiments 1-30 wherein the analyte comprises mRNA.

Embodiment 32. The method of any one of embodiments 1-31 wherein the capture material comprises a nucleotide or nucleoside sequence.

Embodiment 33. The method of embodiment 32 wherein the capture material comprises a poly-T sequence and the analyte comprises RNA.

Embodiment 34. The method of embodiment 33 further comprising synthesizing a cDNA molecule using the RNA analyte as a template.

Embodiment 35. The method of embodiment 34 wherein identifying the analyte comprises sequencing the cDNA.

Embodiment 36. The method of any one of embodiments 1-35 wherein correlating the analyte, the at least partially sequenced perturbation barcodes and spatial index barcodes, or their complement sequences, with the optical imaging comprises identifying the perturbation agent and specific examination area where the cells are present.

Embodiment 37. A method for identifying a biological response of a cell to a perturbation agent, comprising,
a) dispensing cells into a plurality of examination areas comprised on a biochip,
b) dispensing capture microbeads into the plurality of examination areas, the capture microbeads comprising an optical label and a capture material that binds to an analyte;
c) dispensing a perturbation agent and, optionally, perturbation barcodes into the plurality of examination areas;
d) releasing an analyte from the plurality of cells;
e) imaging the biochip to identify the locations of the optical labels;
f) binding the released analyte to the capture material on the capture microbead;
g) identifying the analyte; and
h) correlating the analyte with the optical imaging and spatial location of the capture bead to thereby identify a biological response of a cell to a perturbation agent.

Embodiment 38. The method of embodiment 37 wherein the capture microbead further comprises the perturbation agent and optional perturbation barcodes, and wherein dispensing the perturbation agent and the optional perturbation barcodes comprises releasing the perturbation agent and optional perturbation barcodes from the capture microbead.

Embodiment 39. The method of any one of embodiments 37-38 further comprising that the spatial index microbeads do not comprise a bead type identifier sequence, and wherein the optical label has an emission wavelength of greater than 680 nm.

Embodiment 40. The method of any one of embodiments 37-39 wherein the biochip comprises at least 9,000 examination areas having a volume of 1 nanoliter or greater.

Embodiment 41. The method of any one of embodiments 37-40 wherein the cells are dispensed so that at least 50% of the plurality of examination areas contain a single cell.

Embodiment 42. The method of any one of embodiments 37-41 wherein the capture microbeads do not comprise a bead type identifier (BID) sequence.

Embodiment 43. The method of any one of embodiments 37-42 further comprising incubating the cells in the examination areas for at least 8 hours.

Embodiment 44 The method of any one of embodiments 37-43 wherein at least 50% of the examination areas have more than one cell from the plurality of cells.

Embodiment 45. The method of any one of embodiments 37-44 wherein the optical label comprises a fluorescent dye that comprises a color code.

Embodiment 46. The method of any one of embodiments 37-45 wherein the examination areas on the biochip are identified based on the locations of the color codes from the capture microbeads.

Embodiment 47. The method of any one of embodiments 37-46 wherein the color codes are correlated to a specific examination area on an x-y gradient on the biochip.

Embodiment 48. The method of any one of embodiments 37-46 comprising dispensing at least three capture microbeads into a majority of the examination areas.

Embodiment 49. The method of any one of embodiments 37-48 comprising dispensing more than one cell to each examination area.

Embodiment 50. The method of any one of embodiments 37-49 wherein the perturbation agent is a small molecule drug candidate.

Embodiment 51. The method of any one of embodiments 37-50 wherein the perturbation agent comprises a nucleic acid sequence.

Embodiment 52. The method of any one of embodiments 37-51 wherein the perturbation agent and the perturbation barcode are released from the capture microbead by photocleavage.

Embodiment 53. The method of any one of embodiments 37-52 wherein releasing the analyte from the cells comprises permeabilizing the cells in the plurality of examination areas.

Embodiment 54. The method of any one of embodiments 37-53 wherein the analyte comprises mRNA.

Embodiment 55. The method of embodiment 37-54 further comprising synthesizing a cDNA molecule using the RNA analyte as a template.

Embodiment 56. A method for identifying a biological response of a cell to a perturbation agent, comprising,
a) dispensing cells into a plurality of examination areas comprised on a biochip;
b) dispensing perturbation microbeads into the plurality of examination areas, wherein the perturbation microbeads comprise a perturbation agent and an optical label;
c) dispensing capture microbeads into the plurality of examination areas wherein the capture microbeads comprise a capture material that binds an analyte;
d) imaging the biochip to identify the locations of the optical labels;
e) releasing the perturbation agent from the perturbation microbeads,
f) releasing an analyte from the cells;
g) binding the released analyte to the capture material on the capture microbead;
h) identifying the analyte; and
i) correlating the analyte with the imaging to thereby identify a biological response of a cell to a perturbation agent.

Embodiment 57. The method of embodiment 56 wherein the biochip comprises at least 9,000 examination areas having a volume of 1 nanoliter or greater.

Embodiment 58. The method of any one of embodiments 56-57 wherein the cells are dispensed so that at least 75% of the plurality of examination areas contain a single cell.

Embodiment 59. The method of any one of embodiments 56-58 wherein the perturbation microbeads do not comprise a bead type identifier (BID) sequence.**

Embodiment 60. The method of any one of embodiments 56-59 wherein the method does not involve the sequencing of a bead type identifier sequence.

Embodiment 61. The method of any one of embodiments 56-60 further comprising incubating the cells in the examination areas for at least 8 hours.

Embodiment 62. The method of any one of embodiments 56-61 wherein the perturbation microbeads are selected from a bead library of at least 100 or 250 beads that each have a distinct optical label.

Embodiment 63. The method of any one of embodiments 56-62 wherein the perturbation microbeads comprise fluorescent dyes having an emission peak at a wavelength of 680 um or greater.

Embodiment 64. The method of any one of embodiments 56-63 wherein at least 50% of the examination areas have more than one cell from the plurality of cells.

Embodiment 65. The method of any one of embodiments 56-64 wherein the perturbation microbeads in a well comprise a combination of optical labels that is unique relative to any other examination area in the plurality of examination areas.

Embodiment 66. The method of any one of embodiments 56-65 wherein the perturbation microbeads are chosen from a bead library comprising at least 100 distinct beads.

Embodiment 67. The method of any one of embodiments 56-66 wherein the binding elements and capture material are comprised on a capture group that comprises a unique molecular identifier sequence.

Embodiment 68. The method of any one of embodiments 56-67 wherein the optical label comprises a fluorescent dye that comprises a color code.

Embodiment 69. The method of any one of embodiments 56-68 wherein the examination areas on the biochip are identified based on the locations of the color codes from the perturbation microbeads.

Embodiment 70. The method of any one of embodiments 56-69 wherein the color codes are correlated to a specific examination area on an x-y gradient on the biochip.

Embodiment 71. The method of any one of embodiments 56-70 wherein more than one perturbation microbead is present in at least 50% of the examination areas.

Embodiment 72. The method of any one of embodiments 56-71 comprising dispensing at least three capture microbeads into a majority of the examination areas.

Embodiment 73. The method of any one of embodiments 56-72 wherein the perturbation agent is a small molecule drug candidate.

Embodiment 74. The method of any one of embodiments 56-73 wherein the perturbation agent and, optionally, a perturbation barcode are released from the perturbation microbead by photocleavage.

Embodiment 75. The method of any one of embodiments 56-74 further comprising synthesizing a cDNA molecule using the RNA analyte as a template.

Embodiment 76. A method for identifying a biological response of a cell to a perturbation agent, comprising,
a) dispensing cells into a plurality of examination areas comprised on a biochip, wherein the plurality of examination areas comprise at least one optical label and spatial index barcodes attached to a surface in the plurality of examination areas, and the spatial index barcodes are releasable from the surface in the examination areas,
b) dispensing a perturbation agent and a perturbation barcode sequence into the plurality of examination areas;
c) dispensing capture microbeads into the plurality of examination areas, the capture microbeads comprising a capture material that binds to an analyte, a first binding entity that binds to a perturbation barcode and a second binding entity that binds to a spatial index barcode;
d) imaging the biochip to identify the locations of the optical labels;
e) releasing an analyte from the plurality of cells;
f) releasing the spatial index barcode from the surfaces in the examination areas,
g) binding the perturbation barcode and the spatial-index barcode to their respective binding elements on the capture microbead;
h) binding the released analyte to the capture material;
i) at least partially sequencing the bound perturbation barcode and bound spatial index barcode;
j) identifying the analyte; and
k) correlating the analyte, the at least partially sequenced perturbation barcode and spatial index barcode with the imaging to thereby identify a biological response of a cell to a perturbation agent.

Embodiment 77. The method of embodiment 76 wherein the perturbation barcode and the spatial index barcode are oligonucleotide sequences.

Embodiment 78. The method of any one of embodiments 76-77 wherein the optical label comprises a fluorescent dye.

Embodiment 79. The method of any one of embodiments 76-78 wherein the spatial index barcodes comprise oligonucleotide sequences.

Embodiment 80. The method of any one of embodiments 76-79 wherein the biochip comprises at least 9,000 examination areas having a volume of 1 nanoliter or greater.

Embodiment 81. The method of any one of embodiments 76-80 wherein the cells are dispensed so that at least 75% of the plurality of examination areas contain a single cell.

Embodiment 82. The method of any one of embodiments 76-81 further comprising incubating the cells in the examination areas for at least 8 hours.

Embodiment 83. The method of any one of embodiments 76-82 wherein the optical labels comprise fluorescent dyes having an emission peak at a wavelength of 680 um or greater.

Embodiment 84. The method of any one of embodiments 76-83 wherein at least 50% of the examination areas have more than one cell from the plurality of cells.

Embodiment 85. The method of any one of embodiments 76-84 wherein the examination areas comprise a combination of optical labels that is unique relative to any other examination area in the plurality of examination areas.

Embodiment 86. The method of any one of embodiments 76-85 wherein the spatial-index barcodes are released from the surface in the examination area by photocleavage.

Embodiment 87. A method for identifying a biological response of a cell to a perturbation agent, comprising,
a) dispensing cells into a plurality of examination areas comprised on a biochip;
b) dispensing a perturbation agent and a perturbation barcode into the plurality of examination areas;
c) dispensing capture microbeads into the plurality of examination areas wherein the capture microbeads comprise a capture material that binds to an analyte, a binding element for binding the perturbation barcode, and an optical label;
d) imaging the biochip to identify the locations of the optical labels;
e) releasing an analyte from the plurality of cells;
f) binding the released analyte to the capture material on the capture bead, and binding the perturbation barcode to the binding element on the capture microbead;
g) identifying the analyte and the perturbation barcode; and
h) correlating the analyte and the perturbation barcode with the imaging to thereby identify a biological response of a cell to a perturbation agent.

Embodiment 88. The method of embodiment 87 wherein the perturbation agent and perturbation barcode are comprised on perturbation microbeads, and dispensing comprises releasing the perturbation agent and perturbation barcode from the perturbation microbeads.

Embodiment 89. The method of any one of embodiments 87-88 wherein the biochip comprises at least 9,000 examination areas having a volume of 1 nanoliter or greater.

Embodiment 90. The method of any one of embodiments 87-89 wherein the cells are dispensed so that at least 75% of the plurality of examination areas contain a single cell.

Embodiment 91. The method of any one of embodiments 87-90 wherein the capture microbeads do not comprise a bead type identifier (BID) sequence.**

Embodiment 92. The method of any one of embodiments 87-91 further comprising incubating the cells in the examination areas for at least 8 hours.

Embodiment 93. The method of any one of embodiments 87-92 wherein the capture microbeads are selected from a bead library of at least 100 or 250 beads that each have a distinct optical label.

Embodiment 94. The method of any one of embodiments 87-93 wherein the capture microbeads comprise fluorescent dyes having an emission peak at a wavelength of 680 um or greater.

Embodiment 95. The method of any one of embodiments 87-94 wherein the capture microbeads are chosen from a bead library comprising at least 100 distinct beads.

Embodiment 96. The method of any one of embodiments 87-95 wherein the barcodes are oligonucleotides and the binding elements and capture material are comprised on a capture group that comprises a unique molecular identifier sequence.

Embodiment 97. The method of any one of embodiments 87-96 wherein the optical label comprises a fluorescent dye that comprises a color code.

Embodiment 98. The method of any one of embodiments 87-97 wherein the examination areas on the biochip are identified based on the locations of the color codes from the capture microbeads.

Embodiment 99. The method of any one of embodiments 87-98 wherein the perturbation agent is a small molecule drug candidate.

Embodiment 100. The method of any one of embodiments 87-99 wherein the perturbation agent and the perturbation barcode are released from the perturbation microbead by photocleavage.

Embodiment 101. The method of embodiment any one of embodiments 87-100 further comprising synthesizing a cDNA molecule using the RNA analyte as a template.

Embodiment 102. A biochip for performing a molecular biology assay, comprising,
a) cells comprised in a plurality of examination areas,
b) perturbation microbeads comprised in the plurality of examination areas wherein the perturbation microbeads comprise a perturbation agent and a perturbation barcode,
c) spatial index microbeads comprised in the plurality of examination areas, wherein more than one spatial index microbead is present in a plurality of the examination areas that contain a cell, wherein the spatial index microbeads comprise an optical label and a spatial-index barcode sequence, and the spatial-index barcode sequence is releasable from the spatial index microbeads, and
d) capture microbeads comprised in the plurality of examination areas, the capture microbeads comprising a binding element that binds to the perturbation barcode and a binding element that binds to the spatial index barcode sequence, and further comprising a capture material that binds to an analyte.

Embodiment 103. The biochip of embodiment 102 wherein at least 50% of the plurality of examination areas contain at least one cell, and wherein the biochip comprises at least 9,000 examination areas.

Embodiment 104. A cell loading device comprising:
a loading chip comprising a plurality of loading wells for loading a plurality of samples onto a biochip;
a biochip comprising a plurality of examination areas for receiving a plurality of samples from the loading wells of the loading chip;
a biochip holder for holding the biochip;
a cover component;
wherein at least one of the biochip holder or cover comprises at least one alignment pin that projects from the biochip holder or cover, and the other of the biochip holder or cover comprising at least one port for receiving and securing the at least one alignment pin;
wherein when the alignment pins are engaged in the ports the plurality of loading wells on the loading chip are in vertical alignment with the plurality of examination areas on the biochip.

Embodiment 105. The device of embodiment 104 wherein the cover component comprises at least two alignment pins, and the biochip holder comprises at least two corresponding ports.

Embodiment 106. The device of any one of embodiments 104-105 further comprising a holding component for holding the loading chip in a fixed position.

Embodiment 107. The device of any one of embodiments 104-106 further comprising at least one inlet tube that fluidly connects the exterior of the device to the biochip and that permits the flow of fluid to the loading chip, and at least one outlet tube that fluidly connects the biochip to the exterior of the biochip and permits the flow of fluid from the loading chip.

Embodiment 108. The device of any one of embodiments 104-107 wherein the at least one inlet tube and at least one outlet tube are comprised on the cover component.

Embodiment 109. The device of any one of embodiments 104-108 wherein the biochip holder holds the biochip in a fixed position, and the holding component holds the loading chip in a fixed position.

Embodiment 110. The device of any one of embodiments 104-109 wherein the holding component is configured to hold the loading chip in a fixed position, and the cover is configured to hold the holding component in a fixed position.

Embodiment 111. The device of any one of embodiments 104-110 wherein the examination areas of the biochip have at least 2× the volume of the loading wells of the loading chip.

What is claimed is:

1. A method for identifying a biological response of a cell to a perturbation agent, comprising,
a) dispensing cells, comprising the cell, into a plurality of examination areas comprised on a biochip,
b) dispensing perturbation agents, comprising the perturbation agent, and perturbation barcodes into the plurality of examination areas,
c) dispensing spatial index microbeads into the plurality of examination areas, wherein the spatial index microbeads comprise optical labels and spatial-index barcodes that are releasable from the spatial index microbeads, and the spatial index microbeads do not comprise a bead type identifier sequence;
d) dispensing capture microbeads into the plurality of examination areas, the capture microbeads comprising binding elements for binding the perturbation barcodes, and binding elements for binding the spatial-index barcodes, and further comprising a capture material that binds to an analyte produced by the cells;
e) imaging the biochip to identify locations of the spatial index microbeads in the biochip;
f) releasing an analyte from the cells;
g) releasing the spatial-index barcodes from the spatial index microbeads,
h) binding the perturbation barcodes and the released spatial-index barcodes to the respective binding elements on the capture microbeads for binding the perturbation barcodes and spatial index barcodes,
i) binding the released analyte to the capture material on the capture microbead;
j) at least partially sequencing the bound perturbation barcodes and the bound spatial-index barcodes, or their complement sequences;
k) identifying the analyte; and
l) correlating the analyte, the at least partially sequenced perturbation barcodes and spatial index barcodes, or their complement sequences, with the identified locations of the spatial index microbeads in the imaged biochip to thereby identify the biological response of the cell to the perturbation agent.

2. The method of claim 1, wherein the perturbation agent and perturbation barcodes are comprised on the capture microbeads or on perturbation microbeads, and the perturbation barcodes comprise an oligonucleotide sequence, and the perturbation agent and barcodes are dispensed by releasing them from the capture microbeads or perturbation microbeads.

3. The method of claim 1, wherein the perturbation agent and perturbation barcodes are comprised on perturbation microbeads, and the perturbation barcodes comprise an oligonucleotide sequence, and the perturbation agent and barcodes are dispensed by releasing them from the perturbation microbeads.

4. The method of claim 1, wherein the spatial index barcodes comprise oligonucleotide sequences.

5. The method of claim 1, wherein the spatial index microbeads are selected from a bead library of at least 100 or 250 beads that each have a distinct optical label.

6. The method of claim 1, wherein the spatial index barcodes comprise an oligonucleotide sequence of no more than 150 nucleotides.

7. The method of claim 1, wherein the spatial index barcodes are oligonucleotides and the binding elements and capture material are comprised on a capture group that comprises a unique molecular identifier sequence.

8. The method of claim 1, wherein the optical labels comprises a fluorescent dye that comprises a color code.

9. The method of claim 1, comprising dispensing more than one cell to each examination area.

10. The method of claim 1, wherein the perturbation agent is a small molecule drug candidate.

11. The method of claim 3, wherein the perturbation agent and the perturbation barcodes are released from the perturbation microbeads by photocleavage.

12. The method of claim 1, wherein the spatial-index barcodes are released from the spatial index microbeads by photocleavage.

13. The method of claim 1, wherein the capture material comprises a poly-T sequence and the analyte comprises RNA.

14. The method of claim 1, wherein correlating the analyte, the at least partially sequenced perturbation barcodes and spatial index barcodes, or their complement sequences, with the identified locations of the spatial index microbeads in the imaged biochip comprises identifying the perturbation agent and specific examination area where the cells are present.

15. A method for identifying a biological response of a cell to a perturbation agent, comprising,
a) dispensing cells, comprising the cell, into a plurality of examination areas comprised on a biochip;
b) dispensing capture microbeads into the plurality of examination areas, the capture microbeads each comprising an optical label and a capture material that binds to an analyte;
c) dispensing perturbation agents, comprising the perturbation agent, and, optionally, perturbation barcodes into the plurality of examination areas;
d) releasing an analyte from the cells;
e) imaging the biochip to identify locations of the optical labels in the biochip;
f) binding the released analyte to the capture material on the capture microbead;
g) identifying the analyte; and
h) correlating the analyte with the imaged biochip and spatial locations of the capture microbeads to thereby identify a biological response of the cell to the perturbation agent.

16. The method of claim 15, wherein the capture microbeads further each comprises the perturbation agents and optional perturbation barcodes, and wherein dispensing the perturbation agents and the optional perturbation barcodes comprises releasing the perturbation agents and optional perturbation barcodes from the capture microbeads.

17. The method of claim 15, wherein the perturbation agent is a small molecule drug candidate.

18. The method of claim 15, wherein the perturbation agent and the perturbation barcodes are released from the capture microbead by photocleavage.

19. A method for identifying a biological response of a cell to a perturbation agent, comprising,
a) dispensing cells into a plurality of examination areas comprised on a biochip;
b) dispensing perturbation microbeads into the plurality of examination areas, wherein the perturbation microbeads comprise perturbation agents, comprising the perturbation agent, and corresponding optical labels;
c) dispensing capture microbeads into the plurality of examination areas wherein the capture microbeads comprise a capture material that binds an analyte;
d) imaging the biochip to identify locations of the optical labels in the biochip;
e) releasing the perturbation agents from the perturbation microbeads,
f) releasing an analyte from the cells;
g) binding the released analyte to the capture material on the capture microbead;
h) identifying the analyte; and
i) correlating the analyte with the imaging to thereby identify the biological response of the cell to the perturbation agent.

20. The method of claim 19, wherein the perturbation microbeads in a well comprise a combination of optical labels that is unique relative to any other examination area in the plurality of examination areas.
